SEQUENCE LISTING (5 sequences)

SEQ ID NO: 1 - protein, 953 aa, Homo sapiens
MEEQGHSEMEIIPSESHPHIQLLKSNRELLVTHIRNTQCLVDNLLKNDYFSAEDAEIVCACPTQPDKVRKILDLVQSKGEEVSEFFLYLLQQLADAYVDLRPWLLEIGFSPSLLTQSKVVVNTDPVSRYTQQLRHHLGRDSKFVLCYAQKEELLLEEIYMDTIMELVGFSNESLGSLNSLACLLDHTTGILNEQGETIFILGDAGVGKSMLLQRLQSLWATGRLDAGVKFFFHFRCRMFSCFKESDRLCLQDLLFKHYCYPERDPEEVFAFLLRFPHVALFTFDGLDELHSDLDLSRVPDSSCPWEPAHPLVLLANLLSGKLLKGASKLLTARTGIEVPRQFLRKKVLLRGFSPSHLRAYARRMFPERALQDRLLSQLEANPNLCSLCSVPLFCWIIFRCFQHFRAAFEGSPQLPDCTMTLTDVFLLVTEVHLNRMQPSSLVQRNTRSPVETLHAGRDTLCSLGQVAHRGMEKSLFVFTQEEVQASGLQERDMQLGFLRALPELGPGGDQQSYEFFHLTLQAFFTAFFLVLDDRVGTQELLRFFQEWMPPAGAATTSCYPPFLPFQCLQGSGPAREDLFKNKDHFQFTNLFLCGLLSKAKQKLLRHLVPAAALRRKRKALWAHLFSSLRGYLKSLPRVQVESFNQVQAMPTFIWMLRCIYETQSQKVGQLAARGICANYLKLTYCNACSADCSALSFVLHHFPKRLALDLDNNNLNDYGVRELQPCFSRLTVLRLSVNQITDGGVKVLSEELTKYKIVTYLGLYNNQITDVGARYVTKILDECKGLTHLKLGKNKITSEGGKYLALAVKNSKSISEVGMWGNQVGDEGAKAFAEALRNHPSLTTLSLASNGISTEGGKSLARALQQNTSLEILWLTQNELNDEVAESLAEMLKVNQTLKHLWLIQNQITAKGTAQLADALQSNTGITEICLNGNLIKPEEAKVYEDEKRIICF

SEQ ID NO: 2 - DNA, 4422 bp, Homo sapiens
cccggccccggcgtccccggaccatggcgctctccgggctcttctctagctctcagcggctgcgaagtctgtaaacctggtggccaagtgattgtaagtcaggagactttccttcggtttctgcctttgatggcaagaggtggagattgtggcggcgattacagaaaacgtctgggaagacaagttgctgtttttatgggaatcgcaggcttggaagagacagaagcaattccagaaataaattggaaattgaagatttaaacaatgttgttttaaaacattctaacttcaaagaatgatgccagaaacttaaaaaggggctgcgcagagtagcaggggccctggagggcgcggcctgaatcctgattgcccttctgctgagaggacacacgcagctgaagatgaatttgggaaaagtagccgcttgctactttaactatggaagagcagggccacagtgagatggaaataatcccatcagagtctcacccccacattcaattactgaaaagcaatcgggaacttctggtcactcacatccgcaatactcagtgtctggtggacaacttgctgaagaatgactacttctcggccgaagatgcggagattgtgtgtgcctgccccacccagcctgacaaggtccgcaaaattctggacctggtacagagcaagggcgaggaggtgtccgagttcttcctctacttgctccagcaactcgcagatgcctacgtggacctcaggccttggctgctggagatcggcttctccccttccctgctcactcagagcaaagtcgtggtcaacactgacccagtgagcaggtatacccagcagctgcgacaccatctgggccgtgactccaagttcgtgctgtgctatgcccagaaggaggagctgctgctggaggagatctacatggacaccatcatggagctggttggcttcagcaatgagagcctgggcagcctgaacagcctggcctgcctcctggaccacaccaccggcatcctcaatgagcagggtgagaccatcttcatcctgggtgatgctggggtgggcaagtccatgctgctacagcggctgcagagcctctgggccacgggccggctagacgcaggggtcaaattcttcttccactttcgctgccgcatgttcagctgcttcaaggaaagtgacaggctgtgtctgcaggacctgctcttcaagcactactgctacccagagcgggaccccgaggaggtgtttgccttcctgctgcgcttcccccacgtggccctcttcaccttcgatggcctggacgagctgcactcggacttggacctgagccgtgtgcctgacagctcctgcccctgggagcctgcccaccccctggtcttgctggccaacctgctcagtgggaagctgctcaagggggctagcaagctgctcacagcccgcacaggcatcgaggtcccgcgccagttcctgcggaagaaggtgcttctccggggcttctcccccagccacctgcgcgcctatgccaggaggatgttccccgagcgggccctgcaggaccgcctgctgagccagctggaggccaaccccaacctctgcagcctgtgctctgtgcccctcttctgctggatcatcttccggtgcttccagcacttccgtgctgcctttgaaggctcaccacagctgcccgactgcacgatgaccctgacagatgtcttcctcctggtcactgaggtccatctgaacaggatgcagcccagcagcctggtgcagcggaacacacacagcccagtggagaccctccacgccggccgggacactctgtgctcgctggggcaggtggcccaccggggcatggagaagagcctctttgtcttcacccaggaggaggtgcaggcctccgggctgcaggagagagacatgcagctgggcttcctgcgggctttgccggagctgggccccgggggtgaccagcagtcctatgagtttttccacctcaccctccaggccttctttacagccttcttcctcgtgctggacgacagggtgggcactcaggagctgctcaggttcttccaggagtggatgccccctgcgggggcagcgaccacgtcctgctatcctcccttcctcccgttccagtgcctgcagggcagtggtccggcgcgggaagacctcttcaagaacaaggatcacttccagttcaccaacctcttcctgtgcgggctgttgtccaaagccaaacagaaactcctgcggcatctggtgcccgcggcagccctgaggagaaagcgcaaggccctgtgggcacacctgttttccagcctgcggggctacctgaagagcctgccccgcgttcaggtcgaaagcttcaaccaggtgcaggccatgcccacgttcatctggatgctgcgctgcatctacgagacacagagccagaaggtggggcagctggcggccaggggcatctgcgccaactacctcaagctgacctactgcaacgcctgctcggccgactgcagcgccctctccttcgtcctgcatcacttccccaagcggctggccctagacctagacaacaacaatctcaacgactacggcgtgcgggagctgcagccctgcttcagccgcctcactgttctcagactcagcgtaaaccagatcactgacggtggggtaaaggtgctaagcgaagagctgaccaaatacaaaattgtgacctatttgggtttatacaacaaccagatcaccgatgtcggagccaggtacgtcaccaaaatcctggatgaatgcaaaggcctcacgcatcttaaactgggaaaaaacaaaataacaagtgaaggagggaagtatctcgccctggctgtgaagaacagcaaatcaatctctgaggttgggatgtggggcaatcaagttggggatgaaggagcaaaagccttcgcagaggctctgcggaaccaccccagcttgaccaccctgagtcttgcgtccaacggcatctccacagaaggaggaaagagccttgcgagggccctgcagcagaacacgtctctagaaatactgtggctgacccaaaatgaactcaacgatgaagtggcagagagtttggcagaaatgttgaaagtcaaccagacgttaaagcatttatggcttatccagaatcagatcacagctaaggggactgcccagctggcagatgcgttacagagcaacactggcataacagagatttgcctaaatggaaacctgataaaaccagaggaggccaaagtctatgaagatgagaagcggattatctgtttctgagaggatgctttcctgttcatggggtttttgccctggagcctcagcagcaaatgccactctgggcagtcttttgtgtcagtgtcttaaaggggcctgcgcaggcgggactatcaggagtccactgcctccatgatgcaagccagcttcctgtgcagaaggtctggtcggcaaactccctaagtacccgctacaattctgcagaaaaagaatgtgtcttgcgagctgttgtagttacagtaaatacactgtgaagagactttattgcctattataattatttttatctgaagctagaggaataaagctgtgagcaaacagaggaggccagcctcacctcattccaacacctgccatagggaccaacgggagcgagttggtcaccgctcttttcattgaagagttgaggatgtggcacaaagttggtgccaagcttcttgaataaaacgtgtttgatggattagtattatacctgaaatattttcttccttctcagcactttcccatgtattgatactggtcccacttcacagctggagacaccggagtatgtgcagtgtgggatttgactcctccaaggttttgtggaaagttaatgtcaaggaaaggatgcaccacgggcttttaattttaatcctggagtctcactgtctgctggcaaagatagagaatgccctcagctcttagctggtctaagaatgacgatgccttcaaaatgctgcttccactcagggcttctcctctgctaggctaccctcctctagaaggctgagtaccatgggctacagtgtctggccttgggaagaagtgattctgtccctccaaagaaatagggcatggcttgcccctgtggccctggcatccaaatggctgcttttgtctcccttacctcgtgaagaggggaagtctcttcctgcctcccaagcagctgaagggtgactaaacgggcgccaagactcaggggatcggctgggaactgggccagcagagcatgttggacaccccccaccatggtgggcttgtggtggctgctccatgagggtgggggtgatactactagatcacttgtcctcttgccagctcatttgttaataaaatactgaaaacactaaaaaaaaaaaaaaa

SEQ ID NO: 3 - protein, 953 aa, Artificial Sequence
  A synthetic NOD1 mutant
MEEQGHSEMEIIPSESHPHIQLLKSNRELLVTHIRNTQCLQDNLLKNDYFSAEDAEIVCACPTQPDKVRKILDLVQSKGEEVSEFFLYLLQQLADAYVDLRPWLLEIGFSPSLLTQSKVVVNTDPVSRYTQQLRHHLGRDSKFVLCYAQKEELLLEEIYMDTIMELVGFSNESLGSLNSLACLLDHTTGILNEQGETIFILGDAGVGKSMLLQRLQSLWATGRLDAGVKFFFHFRCRMFSCFKESDRLCLQDLLFKHYCYPERDPEEVFAFLLRFPHVALFTFDGLDELHSDLDLSRVPDSSCPWEPAHPLVLLANLLSGKLLKGASKLLTARTGIEVPRQFLRKKVLLRGFSPSHLRAYARRMFPERALQDRLLSQLEANPNLCSLCSVPLFCWIIFRCFQHFRAAFEGSPQLPDCTMTLTDVFLLVTEVHLNRMQPSSLVQRNTRSPVETLHAGRDTLCSLGQVAHRGMEKSLFVFTQEEVQASGLQERDMQLGFLRALPELGPGGDQQSYEFFHLTLQAFFTAFFLVLDDRVGTQELLRFFQEWMPPAGAATTSCYPPFLPFQCLQGSGPAREDLFKNKDHFQFTNLFLCGLLSKAKQKLLRHLVPAAALRRKRKALWAHLFSSLRGYLKSLPRVQVESFNQVQAMPTFIWMLRCIYETQSQKVGQLAARGICANYLKLTYCNACSADCSALSFVLHHFPKRLALDLDNNNLNDYGVRELQPCFSRLTVLRLSVNQITDGGVKVLSEELTKYKIVTYLGLYNNQITDVGARYVTKILDECKGLTHLKLGKNKITSEGGKYLALAVKNSKSISEVGMWGNQVGDEGAKAFAEALRNHPSLTTLSLASNGISTEGGKSLARALQQNTSLEILWLTQNELNDEVAESLAEMLKVNQTLKHLWLIQNQITAKGTAQLADALQSNTGITEICLNGNLIKPEEAKVYEDEKRIICF

SEQ ID NO: 4 - protein, 233 aa, Homo sapiens [sequence omitted]

SEQ ID NO: 5 - protein, 540 aa, Homo sapiens
MNGEAICSALPTIPYHKLADLRYLSRGASGTVSSARHADWRVQVAVKHLHIHTPLLDSERKDVLREAEILHKARFSYILPILGICNEPEFLGIVTEYMPNGSLNELLHRKTEYPDVAWPLRFRILHEIALGVNYLHNMTPPLLHHDLKTQNILLDNEFHVKIADFGLSKWRMMSLSQSRSSKSAPEGGTIIYMPPENYEPGQKSRASIKHDIYSYAVITWEVLSRKQPFEDVTNPLQIMYSVSQGHRPVINEESLPYDIPHRARMISLIESGWAQNPDERPSFLKCLIELEPVLRTFEEITFLEAVIQLKKTKLQSVSSAIHLCDKKKMELSLNIPVNHGPQEESCGSSQLHENSGSPETSRSLPAPQDNDFLSRKAQDCYFMKLHHCPGNHSWDSTISGSQRAAFCDHKTTPCSSAIINPLSTAGNSERLQPGIAQQWIQSKREDIVNQMTEACLNQSLDALLSRDLIMKEDYELVSTKPTRTSKVRQLLDTTDIQGEEFAKVIVQKLKDNKQMGLQPYPEILVVSRSPSLNLLQNKSM

(19) United States
(12) Patent Application Publication
(10) Pub. No.: US 2006/0194740 A1
(43) Pub. Date: Aug. 31, 2006

(54) NOD1 as an anti-tumor agent

(76) Inventors: Richard J. Ulevitch, Del Mar, CA (US); Jean da Silva, San Diego, CA (US); Jiahuai Han, San Diego, CA (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/361,015

(22) Filed: Feb. 23, 2006

Related U.S. Application Data
(60) Provisional application No. 60/656,175, filed on Feb. 25, 2005.
(60) Provisional application No. 60/752,794, filed on Dec. 22, 2005.

(51) Int. Cl. A61K 48/00; A61K 38/04

(52) U.S. Cl. 514/19; 514/44

(57) ABSTRACT

The invention provides compositions and methods for treating tumors that involve increasing the expression of Nod1 and/or the activity of NOD1.

This application claims benefit of the filing dates of U.S. Provisional Ser. No. 60/656,175, filed Feb. 25, 2005, and U.S. Provisional Ser. No. 60/752,794, filed Dec. 22, 2005, the contents of which are incorporated herein by reference.

### GOVERNMENT FUNDING

The invention described herein was made with United States Government support under Grant Number AI15136 awarded by the National Institutes of Health. The United States Government has certain rights in this invention.

### FIELD OF THE INVENTION

The invention relates to Nod1 and its function in apoptosis of transformed, malignant cells.

### BACKGROUND OF THE INVENTION

Cancer is a disease that afflicts many people and is a leading cause of death in humans and non-human animals. Cancers typically involve uncontrolled division of a few cells that then create many new cells. Accordingly, many anti-cancer drugs are agents that inhibit or stop cell growth. While such chemotherapeutic agents have improved the survival rate of patients having neoplastic diseases, the serious side effects associated with many chemotherapeutic agents limits their usage and undermines the health of patients already weakened by cancer. New agents are therefore needed that exhibit enhanced selectivity for cancer cells or that are capable of controlling proliferation of oncocytes.

One major problem with many anticancer agents is their specificity. An anti-cancer drug needs to distinguish between cells that are cancerous and cells that are not cancerous. However, the vast bulk of anticancer drugs are indiscriminate at this regard. Typically anticancer agents have negative hematological effects (e.g., cessation of mitosis and disintegration of formed elements in marrow and lymphoid tissues), and immunosuppressive action (e.g., depressed cell counts), and can also have a severe impact on epithelial tissues (e.g., intestinal mucosa), reproductive tissues (e.g., impairment of spermatogenesis), and the nervous system. See, e.g., P. Calabresi and B. A. Chabner, In: Goodman and Gilman, The Pharmacological Basis of Therapeutics (Pergamon Press, 8th Edition) (pp. 1209-1216).

What is needed are anticancer agents that can beneficially treat selected tumor types, or preferably a wide variety of tumor types, and that is particularly suitable for invasive tumors. Moreover, while such anticancer agents should be effective, they should also exhibit have little or no toxicity.

### SUMMARY OF THE INVENTION

The invention provides compositions and methods for promoting apoptosis in tumor cells that involve increasing Nod1 expression or NOD1 activity.

Thus, one aspect of the invention is a method of promoting tumor regression in a mammal that involves administering to the mammal an agent that increases Nod1 expression or NOD1 activity. Examples of tumors that can be treated with the methods of the invention include brain, bladder, cervix, colon, gall bladder, kidney, liver, lung, pancreas, ovary, prostate, skin, stomach, or thyroid tumors. In some embodiments, the tumor is an estrogen-sensitive tumor or a breast tumor.

Examples of agents that increase NOD1 activity include peptides having the following sequences: D-Ala-L-Glu-Diaminopimelic acid (γTriDAP), γ-D-glutamy-meso-diaminopimelic acid (iE-DAP), γ-D-Gln-DAP (iQ-DAP), D-Ala-L-Glu-Diaminopimelic acid (γTriDAP), and combinations thereof. These peptides can activate the NOD1 protein, and hence the Nod1-dependent pathway leading to apoptosis. Another example of an agent that can increase NOD1 activity is a NOD1 polypeptide. In some embodiments, the NOD1 polypeptide can be a human NOD1 polypeptide, for example, a human NOD1 polypeptide with SEQ ID NO:1 or SEQ ID NO:3.

One example of an agent that can increase Nod1 expression is a nucleic acid that comprises a segment encoding a NOD1 polypeptide. Examples of sequences for NOD1 polypeptides include SEQ ID NO:1 or SEQ ID NO:3. One example of a nucleic acid segment encoding NOD1 polypeptide comprises SEQ ID NO:2. The nucleic acid can further include a regulatory element, for example, a promoter, enhancer, transcriptional termination signal, or a combination thereof. The nucleic acid can be part of an expression cassette or an expression vector or a gene delivery vehicle.

Additional active ingredients can be administered in conjunction with the agent that increases Nod1 expression or NOD1 activity. For example, an effective amount of tumor necrosis factor α can be administered with such agents. In some embodiments, tumor necrosis factor α can enhance the Nod-dependent apoptotic pathway. In addition, an effective amount of cycloheximide can be administered with the agents at increase Nod1 expression or NOD1 activity. Moreover, one or more chemotherapeutic compounds can be administered in conjunction with the agent.

Examples of chemotherapeutic compounds that may be used in the compositions and methods of the invention include Altretamine, Bleomycin, Busulphan, Calcium Folinate, Capecitabine, Carboplatin, Carmustine, Chlorambucil, Cisplatin, Cladribine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Hydroxyurea, Idarubicin, Ifosfamide, Irinotecan, Liposomal doxorubicin, Lomustine, Melphalan, Mercaptopurine, Methotrexate, Mitomycin, Mitoxantrone, Oxaliplatin, Paclitaxel, Pentostatin, Procarbazine, Raltitrexed, Streptozocin, Tegafur-uracil, Temozolomide, Thiotepa, Tioguanine/Thioguanine, Topotecan, Treosulfan, Vinblastine, Vincristine, Vindesine, Vinorelbine, and a combination thereof.

The agent can be administered locally to the site of the tumor and/or be formulated for sustained release.

Another aspect of the invention is a composition that includes a carrier, a nucleic acid that comprises a segment encoding a NOD1 polypeptide and an effective amount of D-Ala-L-Glu-Diaminopimelic acid (γTriDAP), γ-D-glutamy-meso-diaminopimelic acid (iE-DAP), γ-D-Gln-DAP (iQ-DAP), or D-Ala-L-Glu-Diaminopimelic acid (γTriDAP), wherein the composition is formulated for local administration to a tumor. The NOD1 polypeptide can, for example, include SEQ ID NO:1 or SEQ ID NO:3. An example of a nucleic acid segment that encodes a NOD1 polypeptide is SEQ ID NO:2. The nucleic acid employed in the composition can include a regulatory element, for example, a promoter, enhancer, transcriptional termination signal, or a combination thereof. The nucleic acid can be an expression cassette or an expression vector. The nucleic acid comprises a gene delivery vehicle. The composition of the invention can also include other active ingredients, for example, an effective amount of tumor necrosis factor α or a chemotherapeutic compound. The composition can be formulated for local administration to the site of the tumor and/or be formulated for sustained release.

Another aspect of the invention is a method of promoting apoptosis in breast tumor cells comprising contacting the breast tumor cells with an effective amount of D-Ala-L-Glu-Diaminopimelic acid (γTriDAP).

Another aspect of the invention is a method of promoting apoptosis in estrogen-sensitive tumor cells comprising contacting the breast tumor cells with an effective amount of D-Ala-L-Glu-Diaminopimelic acid (γTriDAP).

### DESCRIPTION OF THE FIGURES

FIG. 1A-C illustrates Nod1 involvement in TNF-induced apoptosis. FIG. 1A provides a schematic diagram of the mutated gene that gave rise to a TNFα-resistant phenotype, and was later identified as a Nod1 mutant bearing a blasticidine (blast) gene insertion. The cell line bearing this Nod1 mutation is the MCF7-C20 cell line. The insertion from the pDisrup retroviral construct was mapped to the Nod1 gene. The junction of blasticidine fused with the Nod1 gene occurred at the 3′ end of the Nod1 gene between leucine-rich region 8 (LRR8) and leucine-rich region 9 (LRR9). Hence, the LRR9 and LRR10 regions are 3′ to the blasticidine insertion. FIG. 1B shows that the NOD1 protein is not present in detectable amounts in MCF-7 C20 cells. Cell extracts from MCF-7 parental (called “wt”) and MCF-7 C20 cells were prepared and either immunoprecipitated with a monoclonal anti-NOD1 antibody (upper panel) or directly loaded onto an SDS-PAGE gel (lower panel), then transferred to PVDF membranes. Blots were analyzed by immunoblotting using the same monoclonal anti-NOD1 antibody. FIG. 1C shows that MCF-7 cells are more resistant to TNF-induced apoptosis than MCF-7 C20 cells. MCF-7 and MCF-7 C20 cells were treated with increasing concentrations of TNF (0-40 ng/ml) for 20 h. Cell viability was determined by propidium iodide (PI) exclusion assay and flow cytometry. The graph shows that MCF-7 C20 cells are significantly more likely to undergo apoptosis.

FIG. 2A-D shows that MCF-7 cells undergo apoptosis upon γTriDAP treatment. FIG. 2A illustrates that NOD1 is needed for γTriDAP-induced cell death. MCF-7 Blasto cells that express normal levels of NOD1, MCF-7 C20 cells that express little or no NOD1, or MCF-7 Nod1 cells that over-express NOD1 were treated with γTriDAP or αTriDAP (50 ug/ml each) in the presence (shaded bars) or absence (open bars) of cycloheximide (CHX) (3 ug/ml) for 48 h. Control assays received medium (Med) instead of γTriDAP or αTriDAP. After the 48 h incubation, cells were harvested and incubated with propidium iodide (PI) (4 ug/ml). Cell viability was measured by flow cytometry analysis. Data shown are representative experiments of at least four independent experiments. FIG. 2B shows the levels of NOD1 expression in MCF-7 Blasto cells that were transfected with vector alone, MCF-7 C20 cells that have a disruption in the endogenous Nod1 gene or MCF-7 Nod1 cells that were engineered to over-express NOD1. Expression of NOD1 in MCF-7 Blasto, MCF-7 C20 and MCF-7 Nod1 cells was analyzed by western blotting using monoclonal anti-NOD1 antibody. FIG. 2C illustrates the morphological changes in γTriDAP-treated MCF-7 Nod1 cells. Cells were seeded in 4-well chamber slides and treated with γTriDAP/cycloheximide (CHX) (panels b, d, f) or CHX alone (panels a, c, e). Cells were stained with DAPI (panels c, d) or TUNEL (panels e, f), fixed and observed under a phase contrast (panels a, b) or fluorescence (panels c-f) microscopes. FIG. 2D shows that γTriDAP-induced apoptosis in MCF-7 Nod1 cells was diminished or abolished by two broad spectrum caspase inhibitors, z-VAD-FMK and Boc-D-FMK. MCF-7 Nod1 cells were pretreated with z-VAD or Boc-D-FMK caspase inhibitors (50 uM each) for 30 min before addition of γTriDAP/CHX for 48 h. Cells were incubated with propidium iodide (PI) and apoptotic cell death was measured by flow cytometry.

FIG. 3 illustrates by western analysis that addition of γTriDAP, but not the inactive control tri-peptide αTriDAP, to MCF-7 cells resulted in proteolytic cleavage of poly(ADP-ribose)polymerase (PARP) and of capases 6, 7, 8 and 9. Cleavage of PARP and various caspases was detected by Western blot analysis of cell MCF-7 Blasto, MCF-7 C20 and MCF-7 Nod1 after stimulation with γTriDAP, αTriDAP or medium (control) in the presence or absence of CHX (0.5 ug/ml) for 24 h. Cells were harvested, subjected to western blotting and PARP, caspases, p20, p41/43 and p35 were detected with antibodies reactive thereof.

FIG. 4A-C show that NOD1 mutant V41Q is responsive to γTriDAP and remains functional in the apoptosis pathway, whereas NOD1 mutant K208R is not responsive to γTriDAP and is not active in the apoptosis pathway. FIG. 4A graphically illustrates the percentage of apoptotic cells in different cell lines after treatment with medium (Med., a control) or γTriDAP. The NOD1 V41Q and K208R mutants were constructed by site-directed mutagenesis. The V41Q mutation is in the CARD domain of NOD1, whereas the K208R mutation is thought to block conformational changes required for oligomerization mediated by the Nod/NBD domain. Constructs encoding these NOD1 V41Q and K208R mutant polypeptides were transfected into MCF-7 C20 cells and apoptotic assays were performed. As shown, the V41Q mutant retains NOD1 activity but the K208R mutant does not. FIG. 4B shows that the expression levels of the NOD1 mutant and wild type polypeptides were substantially identical. FIG. 4C illustrates by western analysis that NOD1 expression in MCF-7 cells is needed for proteolytic cleavage of PARP and of capases 6, 7, 8 and 9. Cleavage of PARP and various caspases was detected by Western blot analysis of MCF-7 C20 cells that do not express NOD1 and of MCF-7 C20 Nod1 cells in which a Nod1 construct has been recombinantly introduced into MCF-7 C20 cells. Cells were stimulated with γTriDAP or medium (control) in the presence or absence of cycloheximide (CHX) (0.5 ug/ml) for 24 h. Cells were then harvested, subjected to western blotting. PARP, caspases, p20, p41/43 and p35 were detected with antibodies reactive therewith.

FIG. 5A-C illustrate that Nod2 does not induce apoptosis in MCF-7 cells. In FIG. 5A, MCF-7 Blasto, MCF-7 Nod1 and MCF-7 Nod2 were treated with the NOD1 ligand γTriDAP or the Nod2 ligand muramyl dipeptide (MDP)(20 ug/ml each) in the presence or absence of CHX for 48 h. Cells were then incubated with PI and apoptotic cell death was measured by flow cytometry. As shown, γTriDAP stimulates apoptosis, but the Nod2 ligand does not. In FIG. 5B, expression of NOD1 and NOD2 was confirmed by Western blot analysis using anti-Myc antibodies for detection of the recombinant proteins. FIG. 5C shows that MCF-7 Nod2 cells respond to MDP as detected by interleukin-8 (IL-8) secretion. MCF-7 Blasto, MCF-7 Nod1 and MCF-7 Nod2 cells were stimulated with γTriDAP or MDP in the presence or absence of CHX (0.5 ug/ml) for 24 h. Cell supernatants were then harvested and assayed for IL-8 secretion.

FIG. 6A-D illustrate that caspase 8 and caspase 9 are required for γTriDAP-induced apoptosis. FIG. 6A shows the effects of different caspase inhibitors on γTriDAP-induced apoptosis. MCF-7 Nod1 cells were pretreated with inhibitors of the caspases listed along the x-axis of FIG. 6A for 30 min prior to stimulation with γTriDAP/CHX for 48 h. Cells were then incubated with propidium iodide and cell viability was measured by flow cytometry. All inhibitors were used at a concentration of 100 uM. FIG. 6B shows that a high molecular weight form of NOD1 is detected when Nod1 is co-expressed with caspase 9, indicating that NOD1 interacts with caspase 9. In this experiment, 293 cells were co-transfected with vectors encoding for FLAG-caspase 9 in the presence of empty vector, Myc-NOD1, or Myc-NOD2. Cell extracts were immunoprecipitated (IP) with anti-FLAG antibody and co-precipitated proteins were revealed by immunoblotting (WB) using polyclonal anti-Myc antibody. FIG. 6C shows that CLARP completely prevented γTriDAP-induced apoptosis, whereas Bcl2 inhibited γTriDAP-induced apoptosis only partially. MCF-7 CLARP, MCF-7 CLARP/Nod1, MCF-7 Bcl2 and MCF-7 Bcl2/Nod1 cells were untreated or treated with γTriDAP in the presence or absence of CHX (3 ug/ml) for 48 h. Cells were then incubated with propidium iodide and apoptotic cell death was measured by flow cytometry. FIG. 6D shows western blots illustrating that CLARP, Bcl2 and NOD1 are expressed in MCF-7 cells, indicating that the results observed in FIG. 6C are due to functional differences between CLARP and Bcl2, rather than differences in the expression levels of this two proteins.

FIG. 7A-C illustrate that both wild-type RIP2 and a kinase-deficient (KD) mutant of RIP2 are functional in the NOD1 apoptosis pathway. However, RIP2 lacking the CARD domain acts as a dominant negative inhibitor of NOD1 signaling. FIG. 7A graphically illustrates the percentage of cells that were apoptotic in populations of wild type RIP2, RIP2 KD and RIP2 ΔCARD cells. MCF-7 cells were stably transfected with wild type Myc-RIP2, Myc-RIP2 KD, and Myc-RIP2 ΔCARD and were left untreated or were treated with γTriDAP in the presence or absence of CHX (3 ug/ml) for 48 h. Cells were incubated with PI and apoptotic cell death was measured by flow cytometry. FIG. 7B shows that RIP2 polypeptides were expressed as confirmed by immunoprecipitation of cell extracts with polyclonal anti-Myc antibody and immunoblotting using monoclonal anti-Myc 9E10 antibody. FIG. 7C illustrates γTriDAP-induction of phosphorylation of JNK in RIP2 wild types, MCF-7 RIP2 KD, and RIP2 ΔCARD cells. As shown, exposure of MCF-7 cells expressing wild type RIP2 or RIP2 KD to γTriDAP in the presence of cycloheximide for 2 h induced phosphorylation of JNK. However, no such phosphorylation of JNK was observed in RIP2ΔCARD cells that were treated in the same manner.

FIG. 8A-D illustrate that a synergistic relationship exists between NOD1 and TNFα. FIG. 8A graphically illustrates that the percentage of apoptotic cells increases in dose-specific manner as the concentration of NOD1 increases from 0.0 to 100 μg/ml and the concentration of TNFα increases from 0.5 ng/ml to 1 ng/ml. FIG. 8B graphically illustrates that the percentage of apoptotic cells increases in dose-specific manner as the concentration of cycloheximide increases from 0.0 to 3 μg/ml and the concentration of TNFα increases from 0.5 ng/ml to 1 ng/ml. FIG. 8C graphically illustrates that while γTriDAP increases apoptosis in the presence of TNFα, the inactive control tri-peptide αTriDAP may actually inhibit apoptosis, even at higher doses of TNFα. FIG. 8D illustrates NOD1 expression at various time points after exposure of MCF-7 cells to TNFα.

FIG. 9A-C illustrates NOD1 expression in another human breast cancer cell line, the SKBR3 cancer cell line. FIG. 9A (top) graphically illustrates the percentage of apoptotic SKBR3 wild type and SKBR3 Nod1 cells as a function of TNFα concentration. FIG. 9A (bottom) graphically illustrates the percentage of apoptotic SKBR3 wild type and SKBR3 Nod1 cells as a function of γTriDAP concentration. FIG. 9B (top) graphically illustrates the percentage of apoptotic SKBR3 cells that were observed under different conditions by propidium iodide (PI) or Dioc6. The conditions employed are indicated below the western blots. The abbreviations used are as follows: CHX (cycloheximide), TNF (TNFα), γTri (γTriDAP), αTri (αTriDAP). FIG. 9B2 provides a western analysis illustrating proteolytic cleavage of poly(ADP-ribose)polymerase (PARP) and of capases 3, 7, and 8. Cleavage of PARP and various caspases was detected by Western blot analysis of cell MCF-7 cells after stimulation under the conditions specified below the western blot. Cells were harvested, subjected to western blotting and PARP, caspases, p20, p41/43 and p35 were detected with antibodies reactive thereof. FIG. 9C graphically illustrates the percentage of apoptotic wild type, NOD1-expressing and CLARP-expressing SKBR3 cells that were observed under the conditions indicated below the bar graph. Abbreviations used: CHX (cycloheximide), TNF (TNFα), gTri (γTriDAP), gTC (γTriDAP+CHX), aTC (αTriDAP+CHX).

FIGS. 10A, B and C provide images of mice inoculated with wild type MCF-7 breast cancer cells, NOD1 knockout MCF-7 cells and NOD1-transfected MCF-7 cells, respectively. Mice were inoculated subcutaneously with 3×10⁶ human breast cancer cells. The arrowheads indicated the subcutaneous tumors, which are shown in the close-up images to the right of FIG. 10B.

FIG. 11A-D illustrate that the presence of Nod1 prevents tumor growth in SCID mice. FIG. 11A graphically illustrates the tumor volume in mice injected with MCF-7 C20 (left) and MCF-7 C20/Nod1 (right) cells as a function of time. The cells (3×10⁶ cells/mouse) were injected into the flanks of female SCID mice. Tumor size was measured once a week and volume was determined according to the formula (W2×L)/2. Each line represents tumor growth observed in one mouse (n=4). As illustrated, the tumor volume diminished in mice that received Nod1 expressing tumor cells (C20/Nod1). In contrast, the tumor volume increased in mice that received tumor cells that did not express Nod1 (C20 cells). FIG. 11B graphically illustrates that implanting estrogen pellets into mice prior to injection of MCF-7 Blasto, MCF-7 C20 and MCF-7 C20/Nod1 cells (3×10⁶ cells/mouse) increases tumor growth except when the tumor cells express Nod1 (the C20/Nod1 cells). The shaded bar represents results obtained for mice that received estrogen pellets while the open bar represents results obtained for mice that did not receive estrogen pellets. The tumor volume was determined as described above. FIG. 11C graphically illustrates the tumor volume over time in mice injected with MCF-7 Blasto, MCF-7 C20 and MCF-7 RIP2ΔCARD cells. As shown, tumor volume in mice receiving MCF-7 RIP2ΔCARD cells (*) was greater than in mice that express some Nod1 (Blasto, filled diamonds), but less than in mice that do not express Nod1 (C20, filled squares). FIG. 11D graphically illustrates tumor cell growth as a function of estrogen concentration. As shown, tumor cells that do not express Nod1 (the MCF-7 C20 cells) and tumor cells that express mutant RIP2 (the MCF-7 RIP2ΔCARD cells) are more sensitive to estrogen and exhibit increased cell growth. MCF-7 Blasto, MCF-7 C20, MCF-7 C20/Nod1 and MCF-7 RIP2ΔCARD cells were exposed to increasing concentrations of 17β-estradiol, pulsed with ³H-thimidine and cell growth was determined by liquid scintillation. Data are representative of at least 3 independent experiments.

FIG. 12A-C illustrates that estrogen increases γTriDAP-induced apoptosis and tamoxifen inhibits γTriDAP-induced apoptosis in various MCF-7 tumor cell lines. FIG. 12A graphically illustrates the percent apoptosis in MCF-7 Blasto, MCF-7 C20 and MCF-7 C20/Nod1 cells cultured in increasing amounts or estrogen and either cycloheximide (CHX) or CHX plus γTriDAP (γTri). FIG. 12B graphically illustrates the percent apoptosis in MCF-7 Blasto, MCF-7 C20 and MCF-7 C20/Nod1 cells cultured in increasing amounts of tamoxifen and either cycloheximide (CHX) or CHX plus γTriDAP (γTri). For the estrogen studies, MCF-7 cells were cultured in charcoal-treated medium and stimulated with γTriDAP/CHX in the presence of increasing concentrations of estrogen (E2) and cell viability was measured by propidium iodide (PI) exclusion. For the tamoxifen studies, cells were treated with tamoxifen prior to addition of γTriDAP/CHX and cell viability measured by PI exclusion. FIG. 12C shows immunoblots of cell lysates from in vitro cultured cells (left) and in vivo tumor cells (right) illustrating that expression of estrogen receptor is reduced in cells that express Nod1. Total protein extracts from MCF-7 Blasto, MCF-7 C20 and MCF-7 C20/Nod1 cells and from cells isolated from tumors were analyzed by immunoblotting using antibody against estrogen receptor (ERα), and ERK2 as loading control.

FIG. 13A-D illustrate that RIP2 is an important component of the Nod1 apoptotic pathway. FIG. 13A shows that stable expression of kinase-deficient RIP2 (RIP2 KD) in tumor cell lines sensitizes those cells to apoptotic cell death induced by Nod1 or Nod2 activators. MCF-7 cells were stably transfected with Myc-Nod1, Myc-RIP2 (wild type), Myc-RIP2 KD, and Myc-RIP2 ΔCARD. Test cells were then treated with γTriDAP, αTriDAP and MDP (20 μg/ml each) in the presence or absence of CHX (3 μg/ml) for 48 hr, while control cells were not treated with these agents. Cells were then incubated with propidium iodide (PI) and apoptotic cell death was measured by flow cytometry. FIG. 13B illustrates the effect of TNF upon apoptosis in the same MCF-7 cell lines described in FIG. 13A. These cells lines were treated with TNF (10 ng/ml) for 18 hr and apoptotic cell death was assessed as described for FIG. 13A. TNF increased apoptosis in all the cell lines tested. FIG. 13C shows that MCF-7 RIP2 KD cells exhibited increased γTriDAP-induced apoptosis than MCF-7 Nod1 cells. Cells were incubated in the presence of increasing concentrations of γTriDAP or MDP in the presence of CHX for 48 hr and apoptotic cell death was assessed as described for FIG. 13A. FIG. 13D illustrates IL-8 secretion by MCF-7 Blasto, MCF-7 Nod1 and MCF-7 RIP2 wild type cells after stimulation with γTriDAP in the presence of CHX (0.5 mg/ml) or TNF. After incubation, cell supernatants were harvested and assayed for IL-8 secretion.

### DETAILED DESCRIPTION OF THE INVENTION

According to the present invention, increased Nod1 expression or NOD1 activity leads to tumor regression. The invention therefore involves administering NOD1 polypeptides, Nod1 nucleic acids and agents that increase Nod1 expression or NOD1 activity to subjects as an anti-tumor treatment. In some embodiments, the agents that increase Nod1 expression or activity include peptide activators of NOD1 such as γ-D-glutamy-meso-diaminopimelic acid (iE-DAP), γ-D-Gln-DAP (iQ-DAP), D-Ala-L-Glu-Diaminopimelic acid (γTriDAP), and combinations thereof. While the invention contemplates treating any type of tumor, the compositions and methods of the invention may have particular utility for treating estrogen-sensitive tumors and/or breast cancer tumors.

NOD1

The innate immune system is comprised of families of receptors that recognize components of micro-organisms, viruses, abnormal/damaged host cells and the like, and initiate host responses to eliminate and kill invading organisms or to remove atypical host cells. Recently a family of intracellular, cytosolic proteins known as the Nod/Caterpillar family has been linked to innate immune responses. All members of this family have two conserved domains; the nucleotide-binding oligomerization domain (NBD/NOD) and the carboxyl-terminus leucine-rich repeat (LRR) region. The amino-terminal regions of family members, termed the effector domains, contain variable structures that include caspase recruitment domain (CARD), pyrin, BIR and other domains.

Two members of the Nod/Caterpillar family have been particularly linked to innate immune responses to infection. These members are known as NOD1 (CARD4) and NOD2 (CARD15). The effector domains of NOD1 and NOD2 are made up of one and two CARD domains, respectively. NOD1 and NOD2 were first suggested to be intracellular proteins acting as receptors for bacterial lipopolysaccharide (LPS). Subsequently, it was discovered that the NOD ligands are derived from bacterial peptidoglycan (PGN) and not from LPS. Thus NOD1 and NOD2 are likely to function as intracellular sensors of bacteria or bacterial products during infection. However, studies performed to date indicate that mice deficient in NOD1 or NOD2 do not manifest obvious phenotypes associated with immunodeficiency or increased sensitivity to infection.

Nucleic acid and amino acid sequences for NOD1 and other members of the NOD/Caterpillar family can be found in the art, for example, in the NCBI database. See website at ncbi.nlm.nih.gov. For example, one amino acid sequence for human NOD1 is provided below for easy reference as SEQ ID NO:1 (NCBI accession number Q9Y239; gi: 20137579).

| | |
|---|---|
| 1 | MEEQGHSEME IIPSESHPHI QLLKSNRELL VTHIRNTQCL |
| 41 | VDNLLKNDYF SAEDAEIVCA CPTQPDKVRK ILDLVQSKGE |
| 81 | EVSEFFLYLL QQLADAYVDL RPWLLEIGFS PSLLTQSKVV |
| 121 | VNTDPVSRYT QQLRHHLGRD SKFVLCYAQK EELLLEEIYM |
| 161 | DTIMELVGFS NESLGSLNSL ACLLDHTTGI LNEQGETIFI |
| 201 | LGDAGVGKSM LLQRLQSLWA TGRLDAGVKF FFHFRCRMFS |
| 241 | CFKESDRLCL QDLLFKHYCY PERDPEEVFA FLLRFPHVAL |
| 281 | FTFDGLDELH SDLDLSRVPD SSCPWEPAHP LVLLANLLSG |
| 321 | KLLKGASKLL TARTGIEVPR QFLRKKVLLR GFSPSHLRAY |
| 361 | ARRMFPERAL QDRLLSQLEA NPNLCSLCSV PLFCWIIFRC |
| 401 | FQHFRAAFEG SPQLPDCTMT LTDVFLLVTE VHLNRMQPSS |
| 441 | LVQRNTRSPV ETLHAGRDTL CSLGQVAHRG MEKSLFVFTQ |
| 481 | EEVQASGLQE RDMQLGFLRA LPELGPGGDQ QSYEFFHLTL |
| 521 | QAFFTAFFLV LDDRVGTQEL LRFFQEWMPP AGAATTSCYP |
| 561 | PFLPFQCLQG SGPAREDLFK NKDHFQFTNL FLCGLLSKAK |
| 601 | QKLLRHLVPA AALRRKRKAL WAHLFSSLRG YLKSLPRVQV |
| 641 | ESFNQVQAMP TFIWMLRCIY ETQSQKVGQL AARGICANYL |
| 681 | KLTYCNACSA DCSALSFVLH HFPKRLALDL DNNNLNDYGV |
| 721 | RELQPCFSRL TVLRLSVNQI TDGGVKVLSE ELTKYKIVTY |
| 761 | LGLYNNQITD VGARYVTKIL DECKGLTHLK LGKNKITSEG |
| 801 | GKYLALAVKN SKSISEVGMW GNQVGDEGAK AFAEALRNHP |
| 841 | SLTTLSLASN GISTEGGKSL ARALQQNTSL EILWLTQNEL |
| 881 | NDEVAESLAE MLKVNQTLKH LWLIQNQITA KGTAQLADAL |
| 921 | QSNTGITEIC LNGNLIKPEE AKVYEDEKRI ICF |

A nucleotide sequence for human Nod1 is also available as NCBI accession number BC040339 (gi: 25955660), and reproduced below as SEQ ID NO:2 for easy reference.

| | |
|---|---|
| 1 | CCCGGCCCCG GCGTCCCCGG ACCATGGCGC TCTCCGGGCT |
| 41 | CTTCTCTAGC TCTCAGCGGC TGCGAAGTCT GTAAACCTGG |
| 81 | TGGCCAAGTG ATTGTAAGTC AGGAGACTTT CCTTCGGTTT |
| 121 | CTGCCTTTGA TGGCAAGAGG TGGAGATTGT GGCGGCGATT |
| 161 | ACAGAAAACG TCTGGGAAGA CAAGTTGCTG TTTTTATGGG |
| 201 | AATCGCAGGC TTGGAAGAGA CAGAAGCAAT TCCAGAAATA |
| 241 | AATTGGAAAT TGAAGATTTA AACAATGTTG TTTTAAAACA |
| 281 | TTCTAACTTC AAAGAATGAT GCCAGAAACT TAAAAAGGGG |
| 321 | CTGCGCAGAG TAGCAGGGGC CCTGGAGGGC GCGGCCTGAA |
| 361 | TCCTGATTGC CCTTCTGCTG AGAGGACACA CGCAGCTGAA |
| 401 | GATGAATTTG GGAAAAGTAG CCGCTTGCTA CTTTAACTAT |
| 441 | GGAAGAGCAG GGCCACAGTG AGATGGAAAT AATCCCATCA |
| 481 | GAGTCTCACC CCCACATTCA ATTACTGAAA AGCAATCGGG |
| 521 | AACTTCTGGT CACTCACATC CGCAATACTC AGTGTCTGGT |
| 561 | GGACAACTTG CTGAAGAATG ACTACTTCTC GGCCGAAGAT |
| 601 | GCGGAGATTG TGTGTGCCTG CCCCACCCAG CCTGACAAGG |
| 641 | TCCGCAAAAT TCTGGACCTG GTACAGAGCA AGGGCGAGGA |
| 681 | GGTGTCCGAG TTCTTCCTCT ACTTGCTCCA GCAACTCGCA |
| 721 | GATGCCTACG TGGACCTCAG GCCTTGGCTG CTGGAGATCG |
| 761 | GCTTCTCCCC TTCCCTGCTC ACTCAGAGCA AAGTCGTGGT |
| 801 | CAACACTGAC CCAGTGAGCA GGTATACCCA GCAGCTGCGA |
| 841 | CACCATCTGG GCCGTGACTC CAAGTTCGTG CTGTGCTATG |
| 881 | CCCAGAAGGA GGAGCTGCTG CTGGAGGAGA TCTACATGGA |
| 921 | CACCATCATG GAGCTGGTTG GCTTCAGCAA TGAGAGCCTG |
| 961 | GGCAGCCTGA ACAGCCTGGC CTGCCTCCTG GACCACACCA |
| 1001 | CCGGCATCCT CAATGAGCAG GGTGAGACCA TCTTCATCCT |
| 1041 | GGGTGATGCT GGGGTGGGCA AGTCCATGCT GCTACAGCGG |
| 1081 | CTGCAGAGCC TCTGGGCCAC GGGCCGGCTA GACGCAGGGG |
| 1121 | TCAAATTCTT CTTCCACTTT CGCTGCCGCA TGTTCAGCTG |
| 1161 | CTTCAAGGAA AGTGACAGGC TGTGTCTGCA GGACCTGCTC |
| 1201 | TTCAAGCACT ACTGCTACCC AGAGCGGGAC CCCGAGGAGG |
| 1241 | TGTTTGCCTT CCTGCTGCGC TTCCCCCACG TGGCCCTCTT |
| 1281 | CACCTTCGAT GGCCTGGACG AGCTGCACTC GGACTTGGAC |
| 1321 | CTGAGCCGTG TGCCTGACAG CTCCTGCCCC TGGGAGCCTG |
| 1361 | CCCACCCCCT GGTCTTGCTG GCCAACCTGC TCAGTGGGAA |
| 1401 | GCTGCTCAAG GGGGCTAGCA AGCTGCTCAC AGCCCGCACA |
| 1441 | GGCATCGAGG TCCCGCGCCA GTTCCTGCGG AAGAAGGTGC |
| 1481 | TTCTCCGGGG CTTCTCCCCC AGCCACCTGC GCGCCTATGC |
| 1521 | CAGGAGGATG TTCCCCGAGC GGGCCCTGCA GGACCGCCTG |
| 1561 | CTGAGCCAGC TGGAGGCCAA CCCCAACCTC TGCAGCCTGT |
| 1601 | GCTCTGTGCC CCTCTTCTGC TGGATCATCT TCCGGTGCTT |
| 1641 | CCAGCACTTC CGTGCTGCCT TTGAAGGCTC ACCACAGCTG |
| 1681 | CCCGACTGCA CGATGACCCT GACAGATGTC TTCCTCCTGG |
| 1721 | TCACTGAGGT CCATCTGAAC AGGATGCAGC CCAGCAGCCT |
| 1761 | GGTGCAGCGG AACACACACA GCCCAGTGGA GACCCTCCAC |
| 1801 | GCCGGCCGGG ACACTCTGTG CTCGCTGGGG CAGGTGGCCC |
| 1841 | ACCGGGGCAT GGAGAAGAGC CTCTTTGTCT TCACCCAGGA |
| 1881 | GGAGGTGCAG GCCTCCGGGC TGCAGGAGAG AGACATGCAG |
| 1921 | CTGGGCTTCC TGCGGGCTTT GCCGGAGCTG GGCCCCGGGG |
| 1961 | GTGACCAGCA GTCCTATGAG TTTTTCCACC TCACCCTCCA |
| 2001 | GGCCTTCTTT ACAGCCTTCT TCCTCGTGCT GGACGACAGG |
| 2041 | GTGGGCACTC AGGAGCTGCT CAGGTTCTTC CAGGAGTGGA |
| 2081 | TGCCCCCTGC GGGGGCAGCG ACCACGTCCT GCTATCCTCC |
| 2121 | CTTCCTCCCG TTCCAGTGCC TGCAGGGCAG TGGTCCGGCG |
| 2161 | CGGGAAGACC TCTTCAAGAA CAAGGATCAC TTCCAGTTCA |
| 2201 | CCAACCTCTT CCTGTGCGGG CTGTTGTCCA AAGCCAAACA |
| 2241 | GAAACTCCTG CGGCATCTGG TGCCCGCGGC AGCCCTGAGG |
| 2281 | AGAAAGCGCA AGGCCCTGTG GGCACACCTG TTTTCCAGCC |
| 2321 | TGCGGGGCTA CCTGAAGAGC CTGCCCCGCG TTCAGGTCGA |
| 2361 | AAGCTTCAAC CAGGTGCAGG CCATGCCCAC GTTCATCTGG |
| 2401 | ATGCTGCGCT GCATCTACGA GACACAGAGC CAGAAGGTGG |
| 2441 | GGCAGCTGGC GGCCAGGGGC ATCTGCGCCA ACTACCTCAA |
| 2481 | GCTGACCTAC TGCAACGCCT GCTCGGCCGA CTGCAGCGCC |
| 2521 | CTCTCCTTCG TCCTGCATCA CTTCCCCAAG CGGCTGGCCC |
| 2561 | TAGACCTAGA CAACAACAAT CTCAACGACT ACGGCGTGCG |
| 2601 | GGAGCTGCAG CCCTGCTTCA GCCGCCTCAC TGTTCTCAGA |
| 2641 | CTCAGCGTAA ACCAGATCAC TGACGGTGGG GTAAAGGTGC |
| 2681 | TAAGCGAAGA GCTGACCAAA TACAAAATTG TGACCTATTT |
| 2721 | GGGTTTATAC AACAACCAGA TCACCGATGT CGGAGCCAGG |
| 2761 | TACGTCACCA AAATCCTGGA TGAATGCAAA GGCCTCACGC |
| 2801 | ATCTTAAACT GGGAAAAAAC AAAATAACAA GTGAAGGAGG |
| 2841 | GAAGTATCTC GCCCTGGCTG TGAAGAACAG CAAATCAATC |
| 2881 | TCTGAGGTTG GGATGTGGGG CAATCAAGTT GGGGATGAAG |
| 2921 | GAGCAAAAGC CTTCGCAGAG GCTCTGCGGA ACCACCCCAG |
| 2961 | CTTGACCACC CTGAGTCTTG CGTCCAACGG CATCTCCACA |
| 3001 | GAAGGAGGAA AGAGCCTTGC GAGGGCCCTG CAGCAGAACA |
| 3041 | CGTCTCTAGA AATACTGTGG CTGACCCAAA ATGAACTCAA |
| 3081 | CGATGAAGTG GCAGAGAGTT TGGCAGAAAT GTTGAAAGTC |
| 3121 | AACCAGACGT TAAAGCATTT ATGGCTTATC CAGAATCAGA |
| 3161 | TCACAGCTAA GGGGACTGCC CAGCTGGCAG ATGCGTTACA |
| 3201 | GAGCAACACT GGCATAACAG AGATTTGCCT AAATGGAAAC |
| 3241 | CTGATAAAAC CAGAGGAGGC CAAAGTCTAT GAAGATGAGA |
| 3281 | AGCGGATTAT CTGTTTCTGA GAGGATGCTT TCCTGTTCAT |
| 3321 | GGGGTTTTTG CCCTGGAGCC TCAGCAGCAA ATGCCACTCT |
| 3361 | GGGCAGTCTT TTGTGTCAGT GTCTTAAAGG GGCCTGCGCA |
| 3401 | GGCGGGACTA TCAGGAGTCC ACTGCCTCCA TGATGCAAGC |
| 3441 | CAGCTTCCTG TGCAGAAGGT CTGGTCGGCA AACTCCCTAA |
| 3481 | GTACCCGCTA CAATTCTGCA GAAAAAGAAT GTGTCTTGCG |
| 3521 | AGCTGTTGTA GTTACAGTAA ATACACTGTG AAGAGACTTT |
| 3561 | ATTGCCTATT ATAATTATTT TTATCTGAAG CTAGAGGAAT |
| 3601 | AAAGCTGTGA GCAAACAGAG GAGGCCAGCC TCACCTCATT |
| 3641 | CCAACACCTG CCATAGGGAC CAACGGGAGC GAGTTGGTCA |
| 3681 | CCGCTCTTTT CATTGAAGAG TTGAGGATGT GGCACAAAGT |
| 3721 | TGGTGCCAAG CTTCTTGAAT AAAACGTGTT TGATGGATTA |
| 3761 | GTATTATACC TGAAATATTT TCTTCCTTCT CAGCACTTTC |
| 3801 | CCATGTATTG ATACTGGTCC CACTTCACAG CTGGAGACAC |
| 3841 | CGGAGTATGT GCAGTGTGGG ATTTGACTCC TCCAAGGTTT |
| 3881 | TGTGGAAAGT TAATGTCAAG GAAAGGATGC ACCACGGGCT |
| 3921 | TTTAATTTTA ATCCTGGAGT CTCACTGTCT GCTGGCAAAG |
| 3961 | ATAGAGAATG CCCTCAGCTC TTAGCTGGTC TAAGAATGAC |
| 4001 | GATGCCTTCA AAATGCTGCT TCCACTCAGG GCTTCTCCTC |
| 4041 | TGCTAGGCTA CCCTCCTCTA GAAGGCTGAG TACCATGGGC |
| 4081 | TACAGTGTCT GGCCTTGGGA AGAAGTGATT CTGTCCCTCC |
| 4121 | AAAGAAATAG GGCATGGCTT GCCCCTGTGG CCCTGGCATC |
| 4161 | CAAATGGCTG CTTTTGTCTC CCTTACCTCG TGAAGAGGGG |
| 4201 | AAGTCTCTTC CTGCCTCCCA AGCAGCTGAA GGGTGACTAA |
| 4241 | ACGGGCGCCA AGACTCAGGG GATCGGCTGG GAACTGGGCC |
| 4281 | AGCAGAGCAT GTTGGACACC CCCCACCATG GTGGGCTTGT |
| 4321 | GGTGGCTGCT CCATGAGGGT GGGGGTGATA CTACTAGATC |
| 4361 | ACTTGTCCTC TTGCCAGCTC ATTTGTTAAT AAAATACTGA |
| 4401 | AAACACTAAA AAAAAAAAAA AA |

The NOD1 protein appears to have an important role in bacterial recognition and may function as a specific host pattern recognition receptor in intracellular compartments. Recent studies have shown that NOD1 as essential in host recognition of bacterial peptidoglycan containing diaminopimelic acid (Chamaillard et al., Nature Immunology, DOI:10.1038/ni945, Jun. 8, 2003). The core structure recognized by NOD1 is a dipeptide, γ-D-glutamyl-meso-diaminopimelic acid (also referred to as iE-DAP). This dipeptide known to exist only in limited number of bacteria (Escherichia coli and several gram-positive bacteria, such as Bacillus subtilis and Listeria monocytogenes).

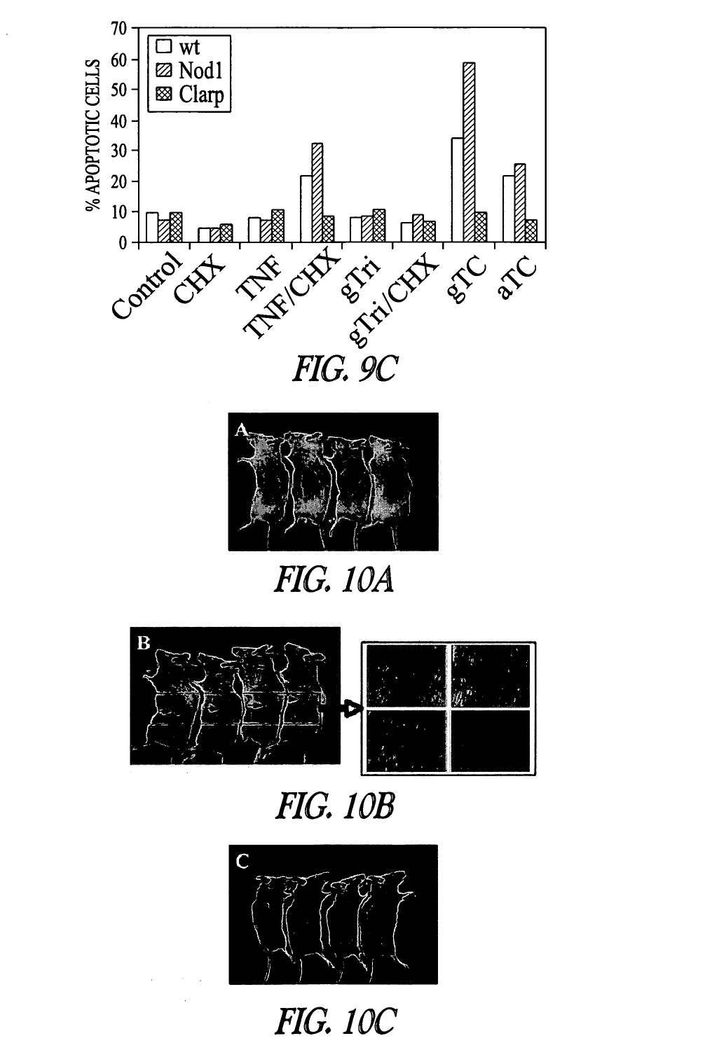

The inventors have discovered that NOD1 sensitizes cells to TNFα-induced apoptosis and that a NOD1-specific ligand induces apoptosis in tumor cells in the absence of any other known apoptotic triggers. In vivo studies using an animal model illustrate and highlight the role that NOD1 has in tumor regression. In particular, as shown herein, xenografts of MCF-7 breast tumor cells placed in SCID mice typically form tumors. However, after a short while these tumors typically regress, even without anti-tumor treatment. But, when Nod1^{-/-} MCF-7 tumor cells are grafted into mice, the tumors do not regress and, instead, continue to grow (see FIG. 10). When NOD1 function is added back to Nod1^{-/-} MCF-7 tumor cells (by transfection of the appropriate genetic construct), tumors generated by grafting these NOD1-expressing cells will now regress. Hence, NOD1 expression can help control tumor cell growth and can lead to apoptosis of tumor cells.

The invention also provides a NOD1 mutant (V41Q) polypeptide that retains apoptosis activity. The sequence of this V41Q mutant NOD1 polypeptide is provided below as SEQ ID NO:3, with the V41Q mutation in bold and underlined.

| | |
|---|---|
| 1 | MEEQGHSEME IIPSESHPHI QLLKSNRELL VTHIRNTQCL |
| 41 | QDNLLKNDYF SAEDAEIVCA CPTQPDKVRK ILDLVQSKGE |
| 81 | EVSEFFLYLL QQLADAYVDL RPWLLEIGFS PSLLTQSKVV |
| 121 | VNTDPVSRYT QQLRHHLGRD SKFVLCYAQK EELLLEEIYM |
| 161 | DTIMELVGFS NESLGSLNSL ACLLDHTTGI LNEQGETIFI |
| 201 | LGDAGVGKSM LLQRLQSLWA TGRLDAGVKF FFHFRCRMFS |
| 241 | CFKESDRLCL QDLLFKHYCY PERDPEEVFA FLLRFPHVAL |
| 281 | FTFDGLDELH SDLDLSRVPD SSCPWEPAHP LVLLANLLSG |
| 321 | KLLKGASKLL TARTGIEVPR QFLRKKVLLR GFSPSHLRAY |
| 361 | ARRMFPERAL QDRLLSQLEA NPNLCSLCSV PLFCWIIFRC |
| 401 | FQHFRAAFEG SPQLPDCTMT LTDVFLLVTE VHLNRMQPSS |
| 441 | LVQRNTRSPV ETLHAGRDTL CSLGQVAHRG MEKSLFVFTQ |
| 481 | EEVQASGLQE RDMQLGFLRA LPELGPGGDQ QSYEFFHLTL |
| 521 | QAFFTAFFLV LDDRVGTQEL LRFFQEWMPP AGAATTSCYP |
| 561 | PFLPFQCLQG SGPAREDLFK NKDHFQFTNL FLCGLLSKAK |
| 601 | QKLLRHLVPA AALRRKRKAL WAHLFSSLRG YLKSLPRVQV |
| 641 | ESFNQVQAMP TFIWMLRCIY ETQSQKVGQL AARGICANYL |
| 681 | KLTYCNACSA DCSALSFVLH HFPKRLALDL DNNNLNDYGV |
| 721 | RELQPCFSRL TVLRLSVNQI TDGGVKVLSE ELTKYKIVTY |
| 761 | LGLYNNQITD VGARYVTKIL DECKGLTHLK LGKNKITSEG |
| 801 | GKYLALAVKN SKSISEVGMW GNQVGDEGAK AFAEALRNHP |
| 841 | SLTTLSLASN GISTEGGKSL ARALQQNTSL EILWLTQNEL |
| 881 | NDEVAESLAE MLKVNQTLKH LWLIQNQITA KGTAQLADAL |
| 921 | QSNTGITEIC LNGNLIKPEE AKVYEDEKRI ICF |

The mutation V41Q occurs in the CARD domain of Nod1 and has previously been reported to disrupt binding of Caspase 9 to Nod1. However, contrary to previous results indicating that the V41Q mutation inhibits Nod1-dependent apoptosis, experiments conducted by the inventors show that the V41Q mutant polypeptide is active in the apoptosis pathway (FIG. 4).

Tumors

The compositions and methods of this invention are useful in the treatment of a variety of cancers and tumors including, but not limited to estrogen-sensitive tumors as well as tumors of the breast, bladder, cervix, colon, gall bladder, kidney, liver, lung, pancreas, ovary, prostate, skin, stomach, thyroid, and the like. In some embodiments the compositions of the invention can be used to treat or prevent carcinomas such as bladder, breast, colon, kidney, liver, lung, including small cell lung cancer, esophagus, gall bladder, ovary, pancreas, stomach, cervix, thyroid, prostate, and skin, including squamous cell carcinoma; hematopoietic tumors of lymphoid lineage, including leukemia, acute lymphocytic leukemia, acute lymphoblastic leukemia, B-cell lymphoma, T-cell-lymphoma, Hodgkin's lymphoma, non-Hodgkin's lymphoma, hairy cell lymphoma and Burkett's lymphoma; hematopoietic tumors of myeloid lineage, including acute and chronic myclogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma and schwannomas; other tumors, including melanoma, seminoma, teratocarcinoma, osteosarcoma, xeroderma pigmentosum, keratoxanthoma, thyroid follicular cancer and Kaposi's sarcoma. In some embodiments, the invention can be used to treat or prevent breast cancers and tumors.

For example, a NOD1 polypeptide, Nod1 nucleic acid, agent that can increase NOD1 expression or activity, or a combination thereof, can be injected into or adjacent to a tumor alone, or in combination with other factors such as TNF, to cause the tumor cells to undergo apoptosis. Accordingly, the compositions and methods of the invention may be used to treat cancer.

Modulating NOD1 Expression and Activity

According to the invention, increased NOD1 expression and/or NOD1 activity promotes regression of tumors. Hence, the invention provides methods for treating and preventing tumor growth in a mammal by administering to the mammal NOD1 polypeptides, Nod1 nucleic acids, agents that increase NOD1 expression and/or activity, or a combination thereof. Agents that increase NOD1 expression or activity include any agent that can increase the transcription, translation or activity of NOD1.

Thus, the incidence of tumor regression can be increased or promoted by administering NOD1 polypeptides (e.g. SEQ ID NO:1), nucleic acids that encode NOD1 polypeptides (e.g. a nucleic acid comprising SEQ ID NO:2). Nucleic acids that encode NOD1 can be placed in an expression cassette and/or maintained in a vector for easy manipulation, expression and replication. Methods for generating a nucleic acid that encodes NOD1 and can express NOD1 polypeptides are described in more detail below.

Agents that increase NOD1 expression or activity include small peptidyl ligands that enhance NOD1 activity. For example, γ-D-glutamy-meso-diaminopimelic acid (iE-DAP), γ-D-Gln-DAP (iQ-DAP), D-Ala-L-Glu-Diaminopimelic acid (γTriDAP), and combinations thereof, can increase NOD1 expression or activity. In some embodiments, only γ-D-glutamy-meso-diaminopimelic acid (iE-DAP), or only γ-D-Gln-DAP (iQ-DAP), or only D-Ala-L-Glu-Diaminopimelic acid (γTriDAP) is used or administered. In other embodiments, combinations of γ-D-glutamy-meso-diaminopimelic acid (iE-DAP), γ-D-Gln-DAP (iQ-DAP), and/or D-Ala-L-Glu-Diaminopimelic acid (γTriDAP) are used or administered.

The present invention further provides a method of modulating NOD1 activity in a subject, comprising providing an agent that is capable of altering a subject's NOD1 activity; and administering the agent to a subject under conditions such that the subject's NOD1 activity is altered. In some embodiments, administering the agent to the subject results in regression of a tumor within the subject. The present invention is not limited to a particular compound. Indeed, a variety of compounds are contemplated including, but not limited to, a peptide comprising D-Ala-L-Glu-Diaminopimelic acid (γTriDAP), glutamine-diaminopimelic acid dipeptide and a peptide comprising a glutamic acid-diaminopimelic acid dipeptides (e.g., iE-DAP, iQ-DAP, an analog of iE-DAP or iQ-DAP, or a small molecule mimetic of iE-DAP or iQ-DAP).

Combination Therapies

The invention contemplates compositions and methods that employ combinations of NOD1-promoting agents with other available anti-tumor therapeutics. Dosages of conventional anti-tumor agents are often kept as low as possible because side effects may be observed at higher dosages. According to the invention, a combination of NOD1 and/or agents that increase NOD1 expression or activity, with available anti-tumor agents may improve the spectrum of cancers against which those anti-tumor agents are effective and reduce the required dosage of those anti-tumor agents. Thus, the invention contemplates combinations of the present NOD1-related agents with one or more anti-tumor or carcinostatic agents. Any anti-tumor and carcinostatic agent available to one of skill in the art can be used with the present NOD1-related agents. However, in some embodiments, the selected anti-tumor or carcinostatic agents have different mechanisms of actions, or operate against somewhat different types of cancers or tumors. For example, the NOD1-related agents of the invention can be combined with a carcinostatic agent or an immune activator to combine the pro-apoptotic effects of NOD1 with the anti-neoplastic effect of the carcinostatis agent and/or the pro-immune responses induced by the immune activator. Further, in some cases, radiotherapy or surgical treatment is performed in addition to these methods to improve the effect of the treatment.

Examples of other chemotherapeutic agents that may be used in conjunction with the NOD1-related agents of the invention include Altretamine, Bleomycin, Busulphan, Calcium Folinate, Capecitabine, Carboplatin, Carmustine, Chlorambucil, Cisplatin, Cladribine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Hydroxyurea, Idarubicin, Ifosfamide, Irinotecan, Liposomal doxorubicin, Lomustine, Melphalan, Mercaptopurine, Methotrexate, Mitomycin, Mitoxantrone, Oxaliplatin, Paclitaxel, Pentostatin, Procarbazine, Raltitrexed, Streptozocin, Tegafur-uracil, Temozolomide, Thiotepa, Tioguanine/Thioguanine, Topotecan, Treosulfan, Vinblastine, Vincristine, Vindesine, Vinorelbine and a combination thereof.

In some embodiments, the NOD1-related agents are administered with one or more hormones. For example, the NOD1-related agents can be administered with one or more androgens, progesterones, estrogens or anti-estrogens. Anti-estrogens act by exerting antagonistic effects on cells or tissues that are responsive to estrogen, or by competing with estrogens for access to receptor sites located on the cell surface. For example, the drugs tamoxifen (brand name: Nolvadex) or Arimidex (Anastrozole), are anti-estrogens that can be used. Tamoxifen has been used in the treatment of breast cancer and to reduce the breast cancer incidence in high-risk women. As shown herein, addition of tamoxifen partially blocked γTriDAP-Nod1 induced cell death. Hence, in some instances tamoxifen may not be used in the NOD1 compositions of the invention. However, in other instances, tamoxifen may be useful when included in a therapeutic regimen that includes administration of NOD1 agents.

In another embodiment, the NOD1-related agents of the invention are administered in conjunction with tumor necrosis factor α (TNFα). TNFα is available commercially, for example, from Pro-Spec Tany TechnoGene Ltd. (Israel). Sequences for tumor necrosis factors can be found in the NCBI database at ncbi.nlm.nih.gov. One example of a sequence for human TNFα is provided below as SEQ ID NO:4.

| | |
|---|---|
| 1 | MSTESMIRDV ELAEEALPKK TGGPQGSRRC LFLSLFSFLI |
| 41 | VAGATTLFCL LHFGVIGPQR EESPRDLSLI SPLAQAVRSS |
| 81 | SRTPSDKPVA HVVANPQAEG QLQWLNRRAN ALLANGVELR |
| 121 | DNQLVVPSEG LYLIYSQVLF KGQGCPSTHV LLTHTISRIA |
| 161 | VSYQTKVNLL SAIKSPCQRE TPEGAEAKPW YEPIYLGGVF |
| 201 | QLEKGDRLSA EINRPDYLDF AESGQVYFGI IAL |

Apoptosis

As described herein, NOD1 can promote apoptosis of tumor cells. To treat or prevent tumor growth, one of skill in the art may choose to employ a combination of anti-tumor agents with the NOD1-related agents described herein. Compositions containing a variety of anti-tumor agents, along with the NOD1-related agents of the invention, can be tested in a variety of ways available to the skilled artisan to ascertain whether those compositions optimally promote tumor regression and/or apoptosis of tumor cells.

For example, apoptosis can be assayed by detecting TUNEL (TdT-mediated dUTP nick-end labeling) labeling of the 3′-OH free end of DNA fragments produced during apoptosis (Gavrieli et al. (1992) J. Cell Biol. 119:493). TUNEL assays generally consist of catalytically adding a nucleotide, which has been conjugated to a chromogen system or to a fluorescent tag, to the 3′-OH end of the 180-bp (base pair) oligomer DNA fragments in order to detect the fragments. The presence of a DNA ladder of 180-bp oligomers is indicative of apoptosis. Procedures to detect cell death based on the TUNEL method are available commercially, e.g., from Boehringer Mannheim (Cell Death Kit) and Oncor (Apoptag Plus).

Another apoptosis marker that is currently available is annexin, sold under the trademark APOPTEST™. The annexin marker is used in the “Apoptosis Detection Kit,” which is also commercially available, for example, from R&D Systems. During apoptosis, a cell membrane's phospholipid asymmetry changes such that the phospholipids are exposed on the outer membrane. Annexins are a homologous group of proteins that bind phospholipids in the presence of calcium. A second reagent can be used in conjunction with the reagent that detects annexin, propidium iodide (PI), which is a DNA binding fluorochrome. When a cell population is exposed to both reagents, apoptotic cells stain positive for annexin and negative for PI, necrotic cells stain positive for both, while live cells stain negative for both. Other methods of testing for apoptosis are known in the art and can be used in the methods of the invention.

Tumor regression can be assessed by using animal models, for example, any animal model available to one of skill in the art or the xenograft model described and illustrated herein.

Xenograft Model

The invention also provides a xenograft model that includes cell lines capable of forming tumors in mice. When mice are inoculated with these xenograft cells, tumors appear. The xenograft cell lines of the invention lack Nod1 function and are sometimes referred to herein as Nod1^{-/-} cells. Surprisingly, cells with an identical genetic background except for the presence of a wild type as opposed to a null Nod1 allele, form tumors that quickly regress. Only the Nod1^{-/-} cells that lack Nod1 function form tumors that continue to grow. According to the invention, these isolated Nod1^{-/-} cells are useful for studying tumors and tumor regression. The Nod1^{-/-} cells of the invention can therefore be used to develop chemotherapeutic agents and to investigate the mechanisms of tumor development.

One example of an isolated Nod1^{-/-} cell line of the invention is the MCF-7 C20 cell line. The inventors have observed more robust tumor growth when the C20 clone was transplanted into male mice. Polymerase chain reaction amplification studies determined that the progesterone receptor was missing in MCF-7 C20 cells and in MCF-7 C20 cells in which a functional Nod1 allele had been introduced recombinantly (i.e., MCF-7 C20Nod1 cells). Further PCR studies revealed that the estrogen receptor alpha was present in each of all the three MCF7 cell types (wild type MCF-7 cells, MCF-7 C20 cells and MCF-7 C20Nod1 cells).

Nod1 Expression Cassettes and Vectors

According to the invention, NOD1 polypeptides can be produced recombinantly and then purified for administration as anti-tumor agents to subjects. In another embodiment, nucleic acids that encode NOD1 can be placed in expression cassettes and/or expression vectors. These Nod1 expression cassettes and expression vectors can also be administered as anti-tumor agents to subjects. Hence, the invention provides Nod1 expression cassettes and Nod1 expression vectors.

Mammalian expression of NOD1 polypeptides can be accomplished as described in Dijkema et al., EMBO J. (1985) 4: 761, Gorman et al., Proc. Natl. Acad. Sci. USA (1982b) 79: 6777, Boshart et al., Cell (1985) 41: 521 and U.S. Pat. No. 4,399,216. Other features of mammalian expression can be facilitated as described in Ham and Wallace, Meth. Enz. (1979) 58: 44, Barnes and Sato, Anal. Biochem. (1980) 102: 255, U.S. Pat. Nos. 4,767,704, 4,657,866, 4,927,762, 4,560,655, WO 90/103430, WO 87/00195, and U.S. Pat. No. RE 30,985. Use of such Nod1 nucleic acids can augment or replace the expression of endogenous Nod1 genes.

Nod1 nucleic acids can be placed within linear or circular molecules. They can be placed within autonomously replicating molecules or within molecules without replication sequences. They can be regulated by their own or by other regulatory sequences, as is known in the art. Nucleic acid constructs encoding NOD1 may include transcriptional regulatory elements, such as a promoter element, an enhancer or UAS element, and a transcriptional terminator signal, for controlling transcription of the Nod1 sequences in the cells.

Nod1 nucleic acids can be used in expression cassettes or gene delivery vehicles, for the purpose of delivering a Nod1 mRNA, a full-length NOD1 protein, a NOD1 fusion protein, a NOD1 polypeptide, or a fragment of a NOD1 polypeptide, into a cell, preferably a eukaryotic cell. According to the present invention, a gene delivery vehicle can be, for example, naked plasmid DNA, a viral expression vector, or a Nod1 nucleic acid of the invention in conjunction with a liposome or a condensing agent.

Nod1 nucleic acids can be introduced into suitable host cells using a variety of techniques that are available in the art, such as transferrin-polycation-mediated DNA transfer, transfection with naked or encapsulated nucleic acids, liposome-mediated DNA transfer, intracellular transportation of DNA-coated latex beads, protoplast fusion, viral infection, electroporation, use of nucleic acid microprojectile procedures and calcium phosphate-mediated transfection.

In one embodiment of the invention, the gene delivery vehicle comprises a promoter and a NOD1-encoding nucleic acid. Examples of promoters that can be used include tissue-specific promoters and promoters that are activated by cellular proliferation, such as the thymidine kinase and thymidylate synthase promoters. Other preferred promoters include promoters that are activated by infection with a virus, such as the α- and β-interferon promoters, and promoters that can be activated by a hormone, such as estrogen. Other promoters that can be used include the Moloney virus LTR, the CMV promoter, and the mouse albumin promoter.

A gene delivery vehicle can comprise viral sequences such as a viral origin of replication or packaging signal. These viral sequences can be selected from viruses such as astrovirus, coronavirus, orthomyxovirus, papovavirus, paramyxovirus, parvovirus, picomavirus, poxvirus, retrovirus, togavirus or adenovirus. In some embodiments, the gene delivery vehicle is a recombinant retroviral vector. Recombinant retroviruses and various uses thereof have been described in numerous references including, for example, Mann et al., Cell 33:153, 1983, Cane and Mulligan, Proc. Nat'l. Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91102805).

Examples of retroviruses that can be utilized include avian leukosis virus (ATCC Nos. VR-535 and VR-247), bovine leukemia virus (VR-1315), murine leukemia virus (MLV), mink-cell focus-inducing virus (Koch et al., J. Vir. 49:828, 1984; and Oliff et al., J. Vir. 48:542, 1983), murine sarcoma virus (ATCC Nos. VR-844, 45010 and 45016), reticuloendotheliosis virus (ATCC Nos. VR-994, VR-770 and 45011), Rous sarcoma virus, Mason-Pfizer monkey virus, baboon endogenous virus, endogenous feline retrovirus (e.g., RD114), and mouse or rat gL30 sequences used as a retroviral vector. Strains of MLV from which recombinant retroviruses can be generated include 4070A and 1504A (Hartley and Rowe, J. Vir. 19:19, 1976), Abelson (ATCC No. VR-999), Friend (ATCC No. VR-245), Graffi (Ru et al., J. Vir. 67:4722, 1993; and Yantchev Neopksma 26:397, 1979), Gross (ATCC No. VR-590), Kirsten (Albino et al., J. Exp. Med. 164:1710, 1986), Harvey sarcoma virus (Manly et al., J. Vir. 62:3540, 1988; and Albino et al., J. Exp. Med. 164:1710, 1986) and Rauscher (ATCC No. VR-998), and Moloney MLV (ATCC No. VR-190). A non-mouse retrovirus that can be used is Rous sarcoma virus, for example, Bratislava (Manly et al., J. Vir. 62:3540, 1988; and Albino et al., J. Exp. Med. 164:1710, 1986), Bryan high titer (e.g., ATCC Nos. VR-334, VR-657, VR-726, VR-659, and VR-728), Bryan standard (ATCC No. VR-140), Carr-Zilber (Adgighitov et al., Neoplasma 27:159, 1980), Engelbreth-Holm (Laurent et al., Biochem Biophys Acta 908:241, 1987), Harris, Prague (e.g., ATCC Nos. VR-772, and 45033), or Schmidt-Ruppin (e.g. ATCC Nos. VR-724, VR-725, VR-354) viruses.

Any of the above retroviruses can be readily utilized in order to assemble or construct retroviral gene delivery vehicles given the disclosure provided herein and standard recombinant techniques (e.g., Sambrook et al., Molecular Cloning: A Laboratory Manual, 2^{nd} Edition (1989), Sambrook et al., Molecular Cloning: A Laboratory Manual, 3^{rd} Edition (2001), and Kunkle, Proc. Natl. Acad. Sci. U.S.A. 82:488, 1985). Portions of retroviral expression vectors can be derived from different retroviruses. For example, retrovirus LTRs can be derived from a murine sarcoma virus, a tRNA binding site from a Rous sarcoma virus, a packaging signal from a murine leukemia virus, and an origin of second strand synthesis from an avian leukosis virus. These recombinant retroviral vectors can be used to generate transduction competent retroviral vector particles by introducing them into appropriate packaging cell lines (see Ser. No. 07/800,921, filed Nov. 29, 1991).

Recombinant retroviruses can be produced that direct the site-specific integration of the recombinant retroviral genome into specific regions of the host cell DNA. Such site-specific integration is useful for mutating or replacing the endogenous NOD1 gene. Site-specific integration can be mediated by a chimeric integrase incorporated into the retroviral particle (see Ser. No. 08/445,466 filed May 22, 1995). It is preferable that the recombinant viral gene delivery vehicle is a replication-defective recombinant virus.

Packaging cell lines suitable for use with the above-described retroviral gene delivery vehicles can be readily prepared (see WO 92/05266) and used to create producer cell lines (also termed vector cell lines or “VCLs”) for production of recombinant viral particles. In some embodiments of the present invention, packaging cell lines are made from human (e.g., HT1080 cells) or mink parent cell lines, thereby allowing production of recombinant retroviral gene delivery vehicles that are capable of surviving inactivation by human serum. The construction of such recombinant retroviral gene delivery vehicles is described in detail in WO 91/02805. These recombinant retroviral gene delivery vehicles can be used to generate transduction competent retroviral particles by introducing them into appropriate packaging cell lines. Similarly, adenovirus gene delivery vehicles can also be readily prepared and utilized given the disclosure provided herein (see also Berkner, Biotechniques 6:616-627, 1988, and Rosenfeld et al., Science 252:431-434, 1991, WO 93/07283, WO 93/06223, and WO 93/07282).

A gene delivery vehicle can also be a recombinant adenoviral gene delivery vehicle. Such vehicles can be readily prepared and utilized given the disclosure provided herein and information available in the art (see, e.g., Berkner, Biotechniques 6:616, 1988, and Rosenfeld et al., Science 252:431, 1991, WO 93/07283, WO 93/06223, and WO 93/07282). Adeno-associated viral gene delivery vehicles can also be constructed and used to deliver proteins or nucleic acids of the invention to cells in vitro or in vivo. The use of adeno-associated viral gene delivery vehicles in vitro is described in Chatteijee et al., Science 258: 1485-1488 (1992), Walsh et al., Proc. Nat'l. Acad. Sci. 89: 7257-7261 (1992), Walsh et al., J. Clin. Invest. 94: 1440-1448 (1994), Flotte et al., J. Biol. Chem. 268: 3781-3790 (1993), Ponnazhagan et al., J. Exp. Med. 179: 733-738 (1994), Miller et al., Proc. Nat'l Acad. Sci. 91: 10183-10187 (1994), Einerhand et al., Gene Ther. 2: 336-343 (1995), Luo et al., Exp. Hematol. 23: 1261-1267 (1995), and Zhou et al., Gene Therapy 3: 223-229 (1996). In vivo use of these vehicles is described in Flotte et al., Proc. Nat'l Acad. Sci. 90: 10613-10617(1993), and Kaplitt et al., Nature Genet. 8:148-153 (1994).

In another embodiment of the invention, a gene delivery vehicle is derived from a togavirus. Such togaviruses include alphaviruses such as those described in U.S. Ser. No. 08/405,627, filed Mar. 15, 1995, WO 95/07994. Alpha viruses, including Sindbis and ELVS viruses can be gene delivery vehicles for nucleic acids of the invention. Alpha viruses are described in WO 94/21792, WO 92/10578 and WO 95/07994. Several different alphavirus gene delivery vehicle systems can be constructed and used to deliver nucleic acids to a cell according to the present invention. Representative examples of such systems include those described in U.S. Pat. Nos. 5,091,309 and 5,217,879. In some embodiments, alphavirus gene delivery vehicles for use in the present invention include those that are described in WO 95/07994.

The recombinant viral vehicle can also be a recombinant alphavirus viral vehicle based on a Sindbis virus. Sindbis constructs, as well as numerous similar constructs, can be readily prepared. Sindbis viral gene delivery vehicles typically comprise a 5′ sequence capable of initiating Sindbis virus transcription, a nucleotide sequence encoding Sindbis non-structural proteins, a viral junction region inactivated so as to prevent fragment transcription, and a Sindbis RNA polymerase recognition sequence. Optionally, the viral junction region can be modified so that nucleic acid transcription is reduced, increased, or maintained. As will be appreciated by those of ordinary skill in the art, corresponding regions from other alphaviruses can be used in place of those described above.

The viral junction region of an alphavirus-derived gene delivery vehicle can comprise a first viral junction region that has been inactivated in order to prevent transcription of the nucleic acid and a second viral junction region that has been modified such that nucleic acid transcription is reduced. An alphavirus-derived vehicle can also include a 5′ promoter capable of initiating synthesis of viral RNA from cDNA and a 3′ sequence that controls transcription termination.

Other recombinant togaviral gene delivery vehicles that can be utilized in the present invention include those derived from Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309 and 5,217,879, as well as in WO 92/10578.

Other viral gene delivery vehicles suitable for use in the present invention include, for example, those derived from poliovirus (Evans et al., Nature 339:385, 1989, and Sabin et al., J. Biol. Standardization 1:115, 1973) (ATCC VR-58); rhinovirus (Arnold et al., J. Cell. Biochem. L401, 1990) (ATCC VR-1110); pox viruses, such as canary pox virus or vaccinia virus (Fisher-Hoch et al., PROC. NATL. ACAD. SCI. U.S.A. 86:317, 1989; Flexner et al., Ann. N.Y. Acad. Sci. 569:86, 1989; Flexner et al., Vaccine 8:17, 1990; U.S. Pat. Nos. 4,603,112 and 4,769,330; WO 89/01973) (ATCC VR-111; ATCC VR-2010); SV40 (Mulligan et al., Nature 277:108, 1979) (ATCC VR-305), (Madzak et al., J. Gen. Vir. 73:1533, 1992); influenza virus (Luytjes et al., Cell 59:1107, 1989; McMicheal et al., The New England Journal of Medicine 309:13, 1983; and Yap et al., Nature 273:238, 1978) (ATCC VR-797); parvovirus such as adeno-associated virus (Samulski et al., J. Vir. 63:3822, 1989, and Mendelson et al., Virology 166:154, 1988) (ATCC VR-645); herpes simplex virus (Kit et al., Adv. Exp. Med. Biol. 215:219, 1989) (ATCC VR-977; ATCC VR-260); Nature 277: 108, 1979); human immunodeficiency virus (EPO 386,882, Buchschacher et al., J. Vir. 66:2731, 1992); measles virus (EPO 440,219) (ATCC VR-24); A (ATCC VR-67; ATCC VR-1247), Aura (ATCC VR-368), Bebaru virus (ATCC VR-600; ATCC VR-1240), Cabassou (ATCC VR-922), Chikungunya virus (ATCC VR-64; ATCC VR-1241), Fort Morgan (ATCC VR-924), Getah virus (ATCC VR-369; ATCC VR-1243), Kyzylagach (ATCC VR-927), Mayaro (ATCC VR-66), Mucambo virus (ATCC VR-580; ATCC VR-1244), Ndumu (ATCC VR-371), Pixuna virus (ATCC VR-372; ATCC VR-1245), Tonate (ATCC VR-925), Triniti (ATCC VR-469), Una (ATCC VR-374), Whataroa (ATCC VR-926), Y-62-33 (ATCC VR-375), O'Nyong virus, Eastern encephalitis virus (ATCC VR-65; ATCC VR-1242), Western encephalitis virus (ATCC VR-70; ATCC VR-1251; ATCC VR-622; ATCC VR-1252), and coronavirus (Hamre et al., Proc. Soc. Exp. Biol. Med. 121:190, 1966) (ATCC VR-740).

A nucleic acid of the invention can also be combined with a condensing agent to form a gene delivery vehicle. In some embodiments, the condensing agent is a polycation, such as polylysine, polyarginine, polyornithine, protamine, spermine, spermidine, and putrescine. Many suitable methods for making linkages between condensing agents and nucleic acids are known in the art (see, for example, Ser. No. 08/366,787, filed Dec. 30, 1994).

In an alternative embodiment, a Nod1 nucleic acid or a Nod1 polypeptide is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell that has internalized the liposome, thereby releasing its contents into the cytoplasm. Prior to interaction with the surface of a cell, however, the liposome membrane acts as a relatively impermeable barrier that sequesters and protects its contents, for example, from degradative enzymes. Additionally, because a liposome is a synthetic structure, specially designed liposomes can be produced that incorporate desirable features. See, Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PROC. NATL. ACAD. SCI. U.S.A. 84: 7851, 1987, Plant et al., Anal. Biochem. 176:420, 1989, and U.S. Pat. No. 4,762,915. Liposomes can encapsulate a variety of nucleic acid and polypeptide molecules including DNA, RNA, plasmids, expression constructs comprising nucleic acids such those disclosed in the present invention, and Nod1 polypeptides.

Liposomal preparations for use in the present invention include cationic (positively charged), anionic (negatively charged) and neutral preparations. Cationic liposomes have been shown to mediate intracellular delivery of plasmid DNA (Felgner et al., Proc. Natl. Acad. Sci. USA 84:7413-7416, 1987), mRNA (Malone et al., Proc. Natl. Acad. Sci. USA 86:6077-6081, 1989), and purified transcription factors (Debs et al, J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin™, from GIBCO BRL, Grand Island, N.Y. See also Feigner et al., Proc. Natl. Acad. Sci. US491: 5148-5152.87, 1994. Other commercially available liposomes include Transfectace (DDAB/DOPE) and DOTAP/DOPE (Boerhinger). Other cationic liposomes can be prepared from readily available materials using techniques available in the art. See, e.g., Szoka et al., Proc. Natl. Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE) and the like. These materials can also be mixed with the DOTMA and DOTAP starting materials in appropriate ratios. Methods for making liposomes using these materials are well known in the art.

The liposomes can comprise multilamellar vesicles (MLVs), small unilamellar vesicles (SUVs), or large unilamellar vesicles (LUVs). The various liposome-nucleic acid complexes are prepared using methods known in the art. See, e.g., Straubinger et al., METHODS OF IMMUNOLOGY (1983), Vol. 101, pp. 512-527; Szoka et al., Proc. Natl. Acad. Sci. USA 87:3410-3414, 1990; Papahadjopoulos et al., Biochim. Biophys. Acta 394:483, 1975; Wilson et al., Cell 17:77, 1979; Deamer and Bangham, Biochim. Biophys. Acta 443:629, 1976; Ostro et al., Biochem. Biophys. Res. Commun. 76:836, 1977; Fraley et al., Proc. Natl. Acad. Sci. USA 76:3348, 1979; Enoch and Strittmatter, Proc. Natl. Acad. Sci. USA 76:145, 1979; Fraley et al., J. Biol. Chem. 255:10431, 1980; Szoka and Papahadjopoulos, Proc. Natl. Acad. Sci. USA 75:145, 1979; and Schaefer-Ridder et al., Science 215:166, 1982.

In addition, lipoproteins can be included with a nucleic acid of the invention for delivery to a cell. Examples of such lipoproteins include chylomicrons, HDL, IDL, LDL, and VLDL. Mutants, fragments, or fusions of these proteins can also be used. Modifications of naturally occurring lipoproteins can also be used, such as acetylated LDL. These lipoproteins can target the delivery of nucleic acids to cells expressing lipoprotein receptors. In some embodiments, if lipoproteins are included with a nucleic acid, no other targeting ligand is included in the composition.

Receptor-mediated targeted delivery of Nod1 nucleic acids to specific tissues can also be used. Receptor-mediated DNA delivery techniques are described in, for example, Findeis et al. (1993), Trends in Biotechnol. 11, 202-05; Chiou et al. (1994), GENE THERAPEUTICS: METHODS AND APPLICATIONS OF DIRECT GENE TRANSFER (J. A. Wolff, ed.); Wu & Wu (1988), J. Biol. Chem. 263, 621-24; Wu et al. (1994), J. Biol. Chem. 269, 542-46; Zenke et al. (1990), Proc. Natl. Acad. Sci. U.S.A. 87, 3655-59; Wu et al. (1991), J. Biol. Chem. 266, 338-42.

In another embodiment, naked nucleic acid molecules are used as gene delivery vehicles, for example, as described in WO 90/11092 and U.S. Pat. No. 5,580,859. Such gene delivery vehicles can be either DNA or RNA and, in certain embodiments, are linked to killed adenovirus. Curiel et al., Hum. Gene. Ther. 3:147-154, 1992. Other suitable vehicles include DNA-ligand (Wu et al., J. Biol. Chem. 264:16985-16987, 1989), lipid-DNA combinations (Feigner et al., Proc. Natl. Acad. Sci. USA 84:7413 7417, 1989), liposomes (Wang et al., Proc. Natl. Acad. Sci. 84:7851-7855, 1987) and microprojectiles (Williams et al., Proc. Natl. Acad. Sci. 88:2726-2730, 1991).

One can increase the efficiency of naked nucleic acid uptake into cells by coating the nucleic acids onto biodegradable latex beads. This approach takes advantage of the observation that latex beads, when incubated with cells in culture, are efficiently transported and concentrated in the perinuclear region of the cells. The beads will then be transported into cells when injected into muscle. Nucleic acid-coated latex beads will be efficiently transported into cells after endocytosis is initiated by the latex beads and thus increase gene transfer and expression efficiency. This method can be improved further by treating the beads to increase their hydrophobicity, thereby facilitating the disruption of the endosome and release of nucleic acids into the cytoplasm.

NOD1-encoding nucleic acids can be introduced into cells in a similar manner. Mechanical methods, such as microinjection, liposome-mediated transfection, electroporation, or calcium phosphate precipitation, can be used to introduce the NOD1 nucleic acid construct into cells promote apoptosis and or tumor cell death. Alternatively, if it is desired that the cells stably retain the DNA construct, the DNA construct can be supplied on a plasmid and maintained as a separate element or integrated into the genome of the cells, as is known in the art.

Expression of an endogenous NOD1 gene in a cell can also be altered by introducing in frame with the endogenous NOD1 gene a DNA construct comprising a NOD1 targeting sequence, a regulatory sequence, an exon, and an unpaired splice donor site by homologous recombination, such that a homologous recombinant cell comprising the DNA construct is formed. The new transcription unit can be used to turn the NOD1 gene on or off as desired. This method of affecting endogenous gene expression is taught in U.S. Pat. No. 5,641,670.

Integration of a delivered NOD1 nucleic acid into the genome of a cell line or tissue can be monitored by any means known in the art. For example, Southern blotting of the delivered NOD1 nucleic acid can be performed. A change in the size of the fragments of a delivered nucleic acid indicates integration. Replication of a delivered nucleic acid can be monitored inter alia by detecting incorporation of labeled nucleotides combined with hybridization to a NOD1 probe. Expression of a NOD1 nucleic acid can be monitored by detecting production of NOD1 mRNA that hybridizes to the delivered nucleic acid or by detecting NOD1 protein. NOD1 protein can be detected immunologically.

RIP2 Modulators

According to the invention, cells are sensitized to apoptosis when “kinase-deficient” RIP2 is expressed in the cells. As used herein, “kinase-deficient” RIP2 means a RIP2 polypeptide that has substantially no kinase function. Hence, the invention provides RIP2 polypeptides without kinase function, RIP2 kinase inhibitors as well as expression cassettes and expression vectors for expression kinase-deficient RIP2.

RIP2 is a serine/threonine kinase that contains a CARD domain at its carboxyl terminus and has been shown to induce NF-κB activation in over-expression systems. RIP2 has also been shown to play a role in regulating both the innate and adaptive immune responses. Mice deficient in Rip2 mounted only an attenuated immune response against Toll-like receptor agonists such as lipopolysaccaride (LPS).

Sequences for RIP2 are available, for example, in the NCBI database. See website at ncbi.nlm.nih.gov. For example, one amino acid sequence for human RIP2 is provided below for easy reference as SEQ ID NO:5 (NCBI accession number AAC27722; gi: 3342910).

| | |
|---|---|
| 1 | MNGEAICSAL PTIPYHKLAD LRYLSRGASG TVSSARHADW |
| 41 | RVQVAVKHLH IHTPLLDSER KDVLREAEIL HKARFSYILP |
| 81 | ILGICNEPEF LGIVTEYMPN GSLNELLHRK TEYPDVAWPL |
| 121 | RFRILHEIAL GVNYLHNMTP PLLHHDLKTQ NILLDNEFHV |
| 161 | KIADFGLSKW RMMSLSQSRS SKSAPEGGTI IYMPPENYEP |
| 201 | GQKSRASIKH DIYSYAVITW EVLSRKQPFE DVTNPLQIMY |
| 241 | SVSQGHRPVI NEESLPYDIP HRARMISLIE SGWAQNPDER |
| 281 | PSFLKCLIEL EPVLRTFEEI TFLEAVIQLK KTKLQSVSSA |
| 321 | IHLCDKKKME LSLNIPVNHG PQEESCGSSQ LHENSGSPET |
| 361 | SRSLPAPQDN DFLSRKAQDC YFMKLHHCPG NHSWDSTISG |
| 401 | SQRAAFCDHK TTPCSSAIIN PLSTAGNSER LQPGIAQQWI |
| 441 | QSKREDIVNQ MTEACLNQSL DALLSRDLIM KEDYELVSTK |
| 481 | PTRTSKVRQL LDTTDIQGEE FAKVIVQKLK DNKQMGLQPY |
| 521 | PEILVVSRSP SLNLLQNKSM |

As indicated above, and described in more detail in the Example, cells expressing kinase-deficient RIP2 are sensitized to apoptosis, when the other domains and functions of RIP2 (e.g. the CARD domain) are largely functional. Therefore, the invention relates to a method of sensitizing cells to apoptosis by contacting the cells with an agent that can inhibit RIP2 kinase. In another embodiment, the invention relates to methods of treating cancer in an animal by administering to the mammal a therapeutically effective amount of a RIP2 kinase inhibitor. The RIP2 kinase inhibitor can also be administered with NOD1 polypeptides or Nod1 nucleic acids, or agents that modulate NOD1 activity.

Any available RIP2 inhibitor may be used in the compositions and methods of the invention. In some embodiments, RIP2 kinase function can be inhibited using p38 inhibitors. For example, p38 inhibitors that can be used to inhibit RIP2 include 2-(4-chlorophenyl)-4-(4-fluorophenyl)-5-pyridin-4-yl-1,2-dihydropyrazol-3-one, SC683 76, SB203580(Iodo), SB202190, SB203580, SB203580 (Sulfone), PD169316, SB220025, SKF-86002, SB239063, or ML 3163. Structures for SB220025, SB203580 and PD169316 are provided below. 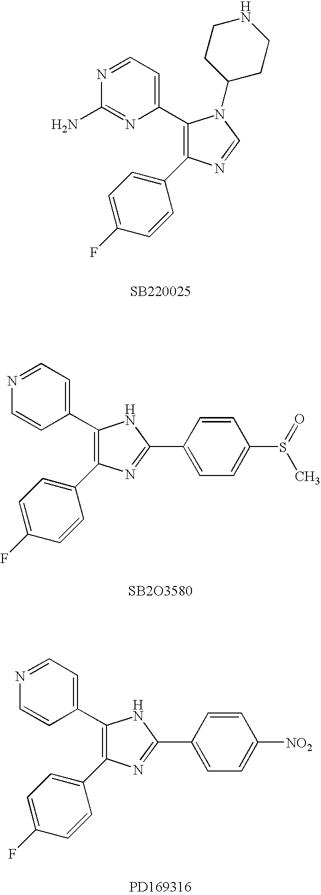
These inhibitors can be used at concentrations comparable to those used to inhibit p38.

In another embodiment, the invention provides a method of identifying a RIP2 inhibitor by observing whether a teat agent can inhibit RIP2 kinase activity. This method can be performed in vitro or in vivo. A control can be included where the RIP2 assay is performed in the absence of the test agent. Decreased activity of RIP2 in the presence of the test agent indicates that the test agent is a RIP2 inhibitor.

Compositions

The NOD1 polypeptides and NOD1 ligands, including their salts, as well as the NOD1 nucleic acids and/or RIP2 kinase inhibitors are administered to promote apoptosis and tumor regression, modulate NOD1 activity or to achieve a reduction in at least one symptom associated with a cancerous condition or other disease associated with inappropriate cellular growth. Other agents can be included as described herein such as anti-tumor agents, chemotherapeutic agents, TNF, RIP2 kinase inhibitors, RIP2 kinase-deficient polypeptides, nucleic acids encoding RIP2 kinase-deficient polypeptides, and the like.

To achieve the desired effect(s), the NOD1 polypeptides, ligands, nucleic acids, and combinations with other agents, may be administered as single or divided dosages. For example, NOD1 polypeptides, ligands and nucleic acids can be administered in dosages of at least about 0.01 mg/kg to about 500 to 750 mg/kg, of at least about 0.01 mg/kg to about 300 to 500 mg/kg, at least about 0.1 mg/kg to about 100 to 300 mg/kg or at least about 1 mg/kg to about 50 to 100 mg/kg of body weight, although other dosages may provide beneficial results. The amount administered will vary depending on various factors including, but not limited to, the polypeptide, ligand or nucleic acid chosen, the disease, the weight, the physical condition, the health, the age of the mammal, whether prevention or treatment is to be achieved, and if the polypeptide, ligand or nucleic acid is chemically modified. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of the polypeptides, ligands, nucleic acids and other agents of the invention may be essentially continuous over a preselected period of time or may be in a series of spaced doses. Both local and systemic administration is contemplated.

To prepare the composition, polypeptides, ligands, nucleic acids and agents are synthesized or otherwise obtained, purified as necessary or desired and then lyophilized and stabilized. The polypeptide, ligand, or nucleic acid can then be adjusted to the appropriate concentration, and optionally combined with other agents. The absolute weight of a given polypeptide, ligand, or nucleic acid included in a unit dose can vary widely. For example, about 0.01 to about 2 g, or about 0.1 to about 500 mg, of at least one polypeptide, nucleic acid or antibody of the invention, or a plurality of polypeptides, ligands, and nucleic acids can be administered. Alternatively, the unit dosage can vary from about 0.01 g to about 50 g, from about 0.01 g to about 35 g, from about 0.1 g to about 25 g, from about 0.5 g to about 12 g, from about 0.5 g to about 8 g, from about 0.5 g to about 4 g, or from about 0.5 g to about 2 g.

Daily doses of the polypeptides, ligands, or nucleic acids of the invention can vary as well. Such daily doses can range, for example, from about 0.1 g/day to about 50 g/day, from about 0.1 g/day to about 25 g/day, from about 0.1 g/day to about 12 g/day, from about 0.5 g/day to about 8 g/day, from about 0.5 g/day to about 4 g/day, and from about 0.5 g/day to about 2 g/day.

Thus, one or more suitable unit dosage forms comprising the therapeutic polypeptides, ligands, or nucleic acids of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. In some embodiments, the NOD1 polypeptides, ligands or nucleic acids are administered locally to tumor or cancer sites.

The therapeutic agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the therapeutic agents with liquid carriers, solid matrices, semi-solid carriers, finely divided solid carriers or combinations thereof, and then, if necessary, introducing or shaping the product into the desired delivery system.

When the therapeutic agents of the invention are prepared for oral administration, they are generally combined with a pharmaceutically acceptable carrier, diluent or excipient to form a pharmaceutical formulation, or unit dosage form. For oral administration, the therapeutic agents may be present as a powder, a granular formulation, a solution, a suspension, an emulsion or in a natural or synthetic polymer or resin for ingestion of the active ingredients from a chewing gum. The therapeutic agents may also be presented as a bolus, electuary or paste. Orally administered therapeutic agents of the invention can also be formulated for sustained release, e.g., the therapeutic agents can be coated, micro-encapsulated, or otherwise placed within a sustained delivery device. The total active ingredients in such formulations comprise from 0.001 to 99.9% by weight of the formulation.

By “pharmaceutically acceptable” is meant a carrier, diluent, excipient, and/or salt that is compatible with the other ingredients of the formulation, and not deleterious to the recipient thereof.

Pharmaceutical formulations containing the therapeutic agents of the invention can be prepared by procedures known in the art using well-known and readily available ingredients. For example, the therapeutic agents can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the therapeutic agents of the invention can include buffering agents such as calcium carbonate, magnesium oxide and magnesium carbonate. Caplets and tablets can also include inactive ingredients such as cellulose, pregelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one therapeutic agent of the invention can contain inactive ingredients such as gelatin, microcrystalline cellulose, sodium lauryl sulfate, starch, talc, and titanium dioxide, and the like, as well as liquid vehicles such as polyethylene glycols (PEGs) and vegetable oil. Moreover, enteric-coated caplets or tablets containing one or more therapeutic agents of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic agents of the invention can also take the form of an aqueous or anhydrous solution or dispersion, or alternatively the form of an emulsion or suspension or salve.

Thus, the therapeutic agents may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The active polypeptides, nucleic acids or antibodies and other ingredients may form suspensions, solutions, or emulsions in oily or aqueous vehicles, and may contain formulatory agents such as suspending, stabilizing and/or dispersing agents. Alternatively, the active polypeptides, nucleic acids or antibodies and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if desired, additional ingredients chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Additionally, the polypeptides, ligands and nucleic acids are well suited to formulation as sustained release dosage forms and the like. The formulations can be so constituted that they release the therapeutic agents, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the therapeutic agents may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention can be delivered via patches or bandages for dermal administration. Alternatively, the polypeptides, ligands and/or nucleic acids can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The therapeutic agents can also be delivered via iontophoresis, e.g., as disclosed in U.S. Pat. No. 4,140,122; 4,383,529; or 4,051,842. The percent by weight of a therapeutic agent of the invention present in a topical formulation will depend on various factors, but generally will be from 0.001% to 95% of the total weight of the formulation, and typically 0.01-85% by weight.

Drops, such as eye drops or nose drops, may be formulated with one or more of the therapeutic agents in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agents may further be formulated for topical administration in the mouth or throat. For example, the active ingredients may be formulated as a lozenge further comprising a flavored base, usually sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The therapeutic agents of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific cancer, tumor, indication or related disease. Any statistically significant attenuation of one or more symptoms of a cancer that has been treated pursuant to the method of the present invention is considered to be a treatment of such cancer within the scope of the invention.

Alternatively, for administration by inhalation or insufflation, the composition may take the form of a dry powder, for example, a powder mix of the therapeutic agents and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in Aerosols and the Lung, Clarke, S. W. and Davia, D. eds., pp. 197-224, Butterworths, London, England, 1984).

Therapeutic agents of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the therapeutic agents of the present invention specific for the indication or disease to be treated. Dry aerosol in the form of finely divided solid polypeptide, ligand, or nucleic acid particles that are not dissolved or suspended in a liquid are also useful in the practice of the present invention. Polypeptides, ligands or nucleic acids of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Pharmoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the active ingredients may also be used in combination with other therapeutic agents, for example, pain relievers, anti-inflammatory agents, antihistamines, antimicrobial agents, bronchodilators and the like, whether for the conditions described or some other condition.

The present invention further pertains to a packaged pharmaceutical composition for modulating Nod1 expression such as a kit or other container. The kit or container holds a therapeutically effective amount of a pharmaceutical composition for modulating Nod1 gene expression, and instructions for using the pharmaceutical composition for modulating Nod1 gene expression. The pharmaceutical composition includes at least one Nod1 nucleic acid of the present invention, in a therapeutically effective amount such that Nod1 gene expression is modulated. The composition can also contain an anti-tumor agent or a chemotherapeutic agent.

In another embodiment, the invention provides a packaged pharmaceutical composition for modulating NOD1 activity. The kit or container holds a therapeutically effective amount of a pharmaceutical composition for modulating NOD1 activity and instructions for using the pharmaceutical composition for modulating NOD1 activity. The pharmaceutical composition includes at least one NOD1 polypeptide or NOD1 ligand of the present invention, in a therapeutically effective amount such that NOD1 activity is modulated.

The invention will be further described by reference to the following detailed examples, which are given for illustration of the invention, and are not intended to be limiting thereof.

### EXAMPLE 1

### NOD1 Induction Leads to Tumor Regression

The Example illustrates that Nod1 sensitizes cells to TNFα-induced apoptosis and that a NOD1-specific ligand induces apoptosis in MCF-7 breast cancer cells in the absence of any other known apoptotic triggers. An in vivo animal model was employed to demonstrate the role of NOD1 in tumor regression that involved a xenograft performed with SCID mice. These data indicate Nod1 plays a key role in controlling tumor cell growth.

### Materials and Methods

Cell culture. Human breast cancer cell lines MCF-7 and SKBR3 were maintained in Dulbecco's modified Eagle's medium supplemented with 10% fetal bovine serum, 2 mM glutamine, 100 U/ml penicillin and 10 μg/ml streptomycin

Mammalian expression constructs and site-directed mutagenesis. Human FLAG-Nod1, FLAG-Nod2 cDNAs were obtained from Dr Gabriel Nuez and are described in Y. Ogura et al., J. Biol. Chem. 276, 4812 (2001). Human Myc-RIP2 wt and Myc-RIP2 (K47A) were a gift from Dr. C. Vincenz (University of Michigan Medical School). Nod1 mutants (V41Q and K208R) were constructed by site-directed mutagenesis. Myc-RIP2 ΔCARD was generated by deletion of the carboxy-terminal CARD domain and cloning into the pcDNA4/Myc/His plasmid (Invitrogen, Carlsbad, Calif.). The nucleotide sequences were all confirmed by DNA sequencing.

Retroviral transfections. The various genes used in this study were cloned into pMSCV-Blasto, pBabe-Puro or pBabe-Neo retroviral vectors. MCF-7 cells were stably transfected using available procedures. Briefly, amphotropic 293 cells were transfected with retroviral vectors encoding selected Nod1, Nod2 and other proteins. Twenty-four hours post-transfection, cells were incubated at 32° C. overnight to produce viral particles. Target cells were infected the next day with virus-containing 293 cell supernatants containing recombinant retroviral particles. Cells were selected with 10 μg/ml blasticidin S (Calbiochem EMD Biosciences Inc., San Diego, Calif.), 500 μg/ml gentamycin (Invitrogen, Carlsbad, Calif.) or 5 μg/ml puromycin (Calbiochem). The expression of all constructs was confirmed by Western blot analysis. To determine the effects of 17β-estradiol (Calbiochem), cells were cultured in phenol red-free DMEM supplemented with % charcoal-stripped FCS. Cells were seeded in 96 well plates with various concentration of 17β-estradiol (E₂) for 24 h and pulsed with ³H-thymidine (1 μCi/well, MP Biomedicals, Irvine, Calif.). Cells were harvested on glass fiber filters and radioactivity measured by liquid scintillation.

Western blot analysis and immunoprecipitation. Cells were washed extensively and lysed using lysis buffer containing 50 mM Hepes, 100 mM NaCl, 2 mM EDTA, 10% glycerol, 1% Nonidet P-40, 14 μM pepstatin A, 100 uM leupeptin, 3 mM benzamidine, 1 mM PMSF, 1 mM sodium pyrophosphate, 10 mM sodium orthovanadate, 100 U/ml aprotinin, and 100 mM sodium fluoride. After incubation for 30 min on ice, cell lysates were centrifuged (14000 rpm, 10 min, 4° C.) and the supernatants were recovered. For immunoprecipitation, cell lysates were mixed with 5 μg of antibody for 3 hour at 4° C. under constant agitation. Immune complexes were allowed to bind to 20 μl protein A-Sepharose beads overnight, beads were washed three times with lysis buffer. Immunoprecipitates were separated on SDS-PAGE and transferred to PVDF membranes.

Cell viability assays. Propidium iodide exclusion assay: Cells were stimulated as indicated for 2 days. Subsequently, cells were harvested, washed twice in FACS buffer (PBS containing 1% FCS and 0.1% NaN₃) and resuspended in propidium iodide (PI)—containing FACS buffer (4 ug/ml). The extent of cell death was analyzed with a FACSCalibur flow cytometer (Becton Dickinson, Mountain View, Calif.). DAPI staining: MCF-7 cells were plated in chamber slides, and stimulated for 2 days. Cells were washed with PBS and apoptotic nuclei were stained with 1 μg/ml DAPI (Sigma Chemical Co., St. Louis, Mo.). Cells were fixed in 4% paraformaldehyde and examined by fluorescence microscopy.

TUNEL staining: Cells were treated with γTriDAP and apoptotic nuclei were monitored by TUNEL (TdT-mediated UTP nick-end labeling) assay according to manufacturer's instruction (ROCHE, Indianapolis, Ind.).

IL-8 ELISA. Concentrations of IL-8 in the culture supernatants of transfected HeLa cells were measured by ELISA using 96-well Immunlon plates (Dynatech Laboratories, Chantilly, Va.). ELISA was performed using the mAb MAB208 for capture and a biotinylated polyclonal rabbit anti-human IL-8 Ab (R&D Systems, Minneapolis, Minn.) followed by streptavidin HRP for detection.

Human xenografts in nude mice. MCF-7 Blasto, MCF-7 C20 and MCF-7 C20/Nod1 cells were trypsinized, washed once with PBS and resuspended to a concentration of 1.5×10⁷/ml. Two hundred microliters of each suspension were inoculated subcutaneously into the flank of athymic SCID/SCID or SCID/Nod (non-obese diabetic) female mice. Tumor size was assessed once a week. Tumor volume was calculated.

### Results

Nod1 has a role in TNFα- and Nod1 ligand-induced apoptosis. The breast cancer epithelial cell line MCF-7 has been widely used to study apoptosis induced by biological signals such as TNFα or by cytotoxic drugs. The MCF-7 cell line has also been used as a model to study estrogen positive breast cancer (Simstein et al., Exp. Biol. Med. 228:995-1003 (2003)).

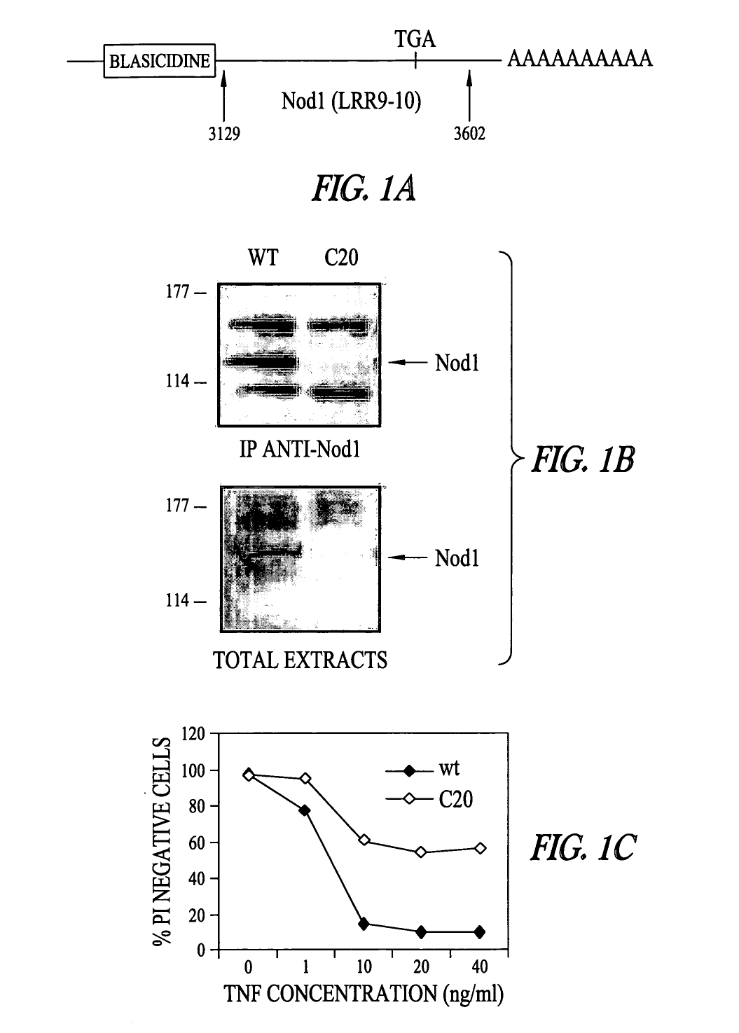

MCF-7 cells were initially used here in a genetic screen employing retrovirus-mediated mutagenesis in order to identify genes required for TNFα-induced cell death. After exposure of MCF-7 cells to the retroviral construct, clones were selected for TNFα resistance and mutated genes were then identified in the TNFα-resistant clones. One of the resistant clones contained a disrupted Nod1 gene; a cell line of this TNFα resistant clone was termed MCF7-C20. The pDisrup insertion from the retroviral construct was mapped to the 3′ portion of the Nod1 gene, in the region of leucine-rich regions 9 and 10 (LRR9-10; see FIG. 1A). This insertion placed a blasticidine open reading frame in the Nod1 coding region. A schematic diagram of the blasticidine-Nod1 fusion mRNA is shown schematically in FIG. 1A. Western blot analysis using an anti-human Nod1 monoclonal antibody was performed to test whether NOD1 protein was expressed in MCF7-C20 cells. Endogenous NOD1 protein was detected in parental MCF-7 cell lysates (labeled “wt”) but western blots of MCF-7 C20 cell lysates failed to reveal detectable expression of Nod1, indicating that the functional Nod1 allele in MCF-7C20 was disrupted (FIG. 1B).

The MCF-7 C20 cell line was deposited under the terms of the Budapest Treaty on or about Feb. 23, 2006 with the American Type Culture Collection (10801 University Blvd., Manassas, Va., 20110-2209 USA (ATCC)) as ATCC Accession No. ATCC Number ______.

Loss of Nod1 function had a significant effect upon apoptosis. In particular, MCF-7 C20 cells (without Nod1 expression) were significantly more resistant to TNFα-induced apoptosis than the parent MCF-7 cells that did express Nod1 (FIG. 1C). These data suggest that Nod1 is as a sensitizer in the TNFα pathway within MCF-7 cells and Nod1 promotes apoptosis in MCF-7 breast cancer cells.

To further investigate the role of Nod1 in apoptosis, a variety of cell types were incubated with the Nod1 ligand, D-Ala-L-Glu-Diaminopimelic acid (γTriDAP), which specifically activates Nod1. The cell types tested included the parental MCF-7 cell line, the Nod1-deficient MCF-7C20 clone, and a cell line engineered to contain an additional human Nod1 allele (MCF-7Nod1) so that Nod1 was over-expressed. To determine whether the resistance of C20 cells to γTriDAP-induced apoptosis resulted from the absence of Nod1, human Nod1 was stably expressed in MCF-7 C20 cells to produce Nod1-sufficient C20 cells (C20/Nod1). As another control, the parental MCF-7 cell line was transfected with an empty retroviral vector to generate MCF-7 Blasto cells. Cells were treated with γTriDAP in the presence or absence of cycloheximide (CHX) and cell death was then determined by propidium iodide staining and flow cytometry.

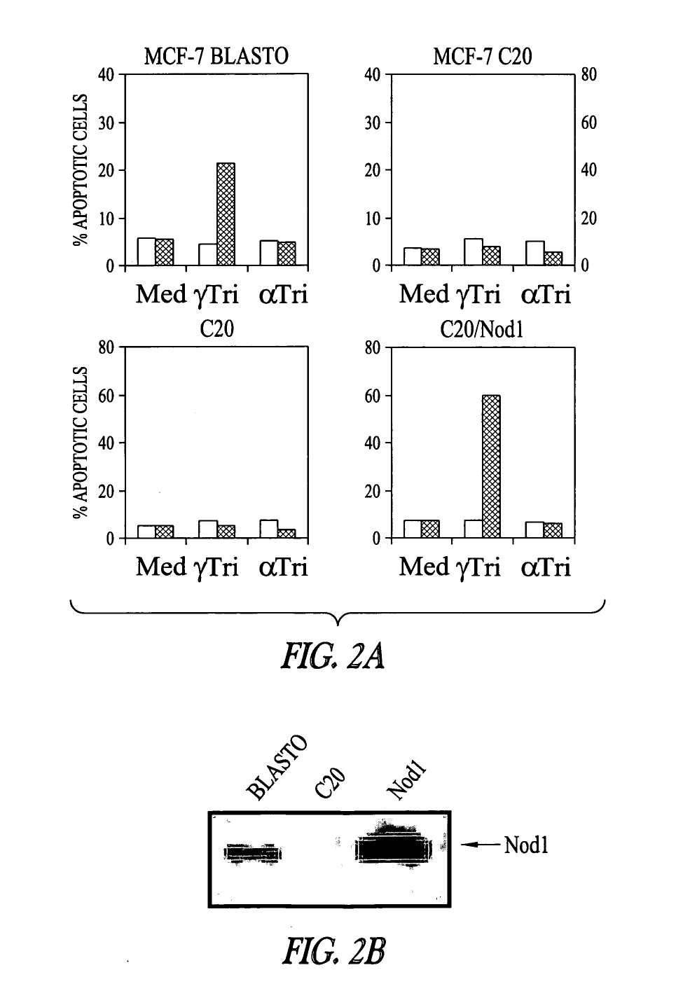

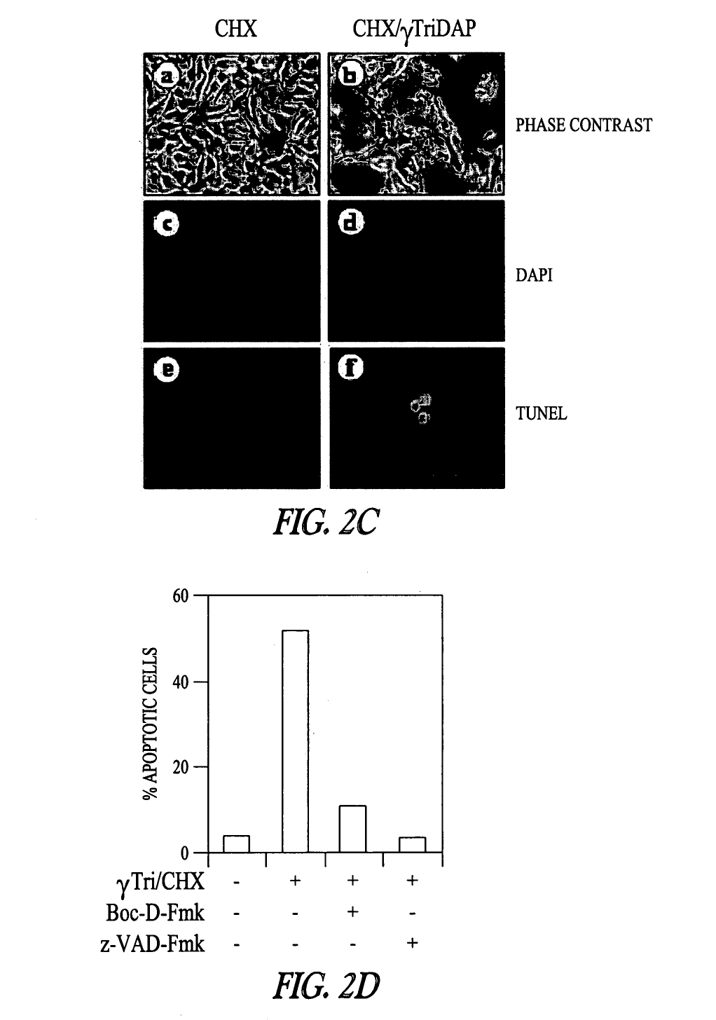

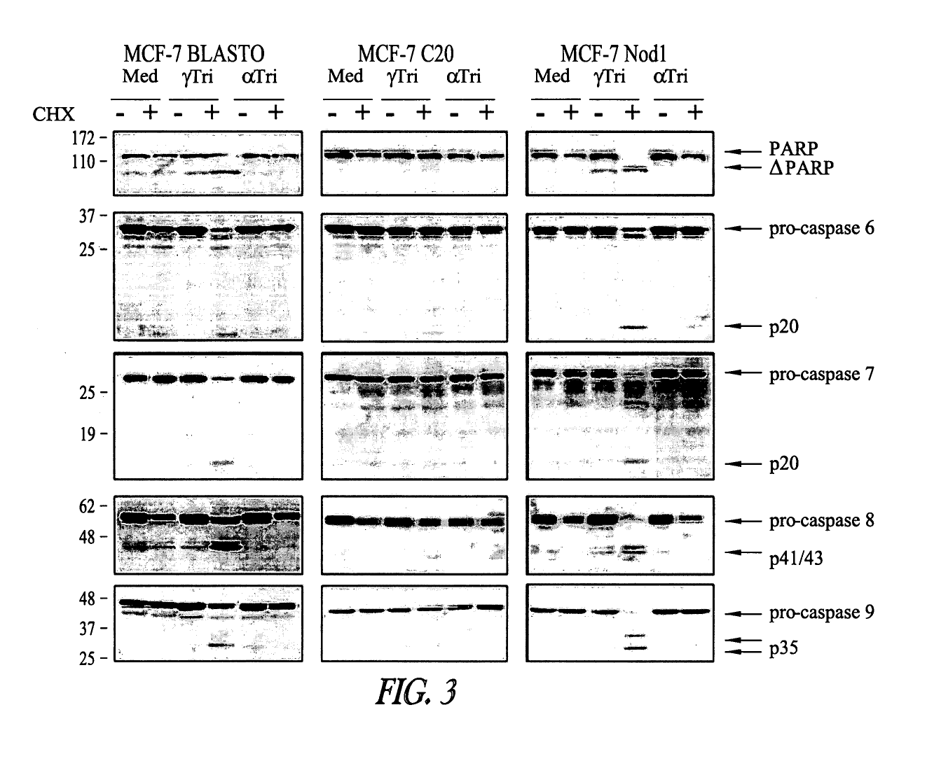

Exposure of wild-type MCF-7 cells to γTriDAP in the presence of cycloheximide induced about 25% cell death (FIG. 2A, upper left panel, shaded bars). No cell death was observed in wild type cells treated with cycloheximide alone (open bars) or γTriDAP alone. In contrast, MCF-7C20 (without Nod1 expression) cells were totally resistant to the effects of γTriDAP plus cycloheximide and substantially no increase in apoptosis was observed in these cells (FIG. 2A, upper right panel). Reintroduction of Nod1 into MCF-7C20 cells restored full sensitivity of these cells to γTriDAP-induced apoptosis (FIG. 2A, lower right panel). The combination of cycloheximide and γTriDAP induced extensive cell death in MCF-7 cells that stably over-expressed Nod1—cell death in these MCF-7Nod1 cells reached almost 60% after 48 h treatment (FIG. 2A, lower right panel).

The Nod1 ligand, γTriDAP was needed for optimal induction of apoptosis. An inactive control tri-peptide named αTriDAP, where mesoDAP was bound to Glu in the a position rather than in the γ position as in γTriDAP, did not induce cell death when incubated with MCF-7 cells in the presence of cycloheximide (αTri in FIG. 2A).

As a control, medium (“Med”) was used instead of γTriDAP or αTriDAP. Addition of medium had substantially no effect on apoptosis. Expression of endogenous Nod1, and over-expression of Nod1, in the MCF-7 Blasto and MCF-7Nod1 cells was confirmed by immunoprecipitation and Western blot analysis (FIG. 2B). No such expression was seen in MCF-7 C20 cells (FIG. 2B). These data show that Nod1 in the presence of γTriDAP sensitizes MCF-7 breast cancer cells to apoptosis.

However, treatment of cells with γTriDAP alone did not induce apoptosis in wild type cells. Instead, addition of both γTriDAP and cycloheximide was needed before apoptosis was observed. Pro-apoptotic agents such as cycloheximide are frequently needed before apoptosis is observed. However, the changes in sensitivity to γTriDAP or TNFα that were noted with MCF-7 cells were not seen with other apoptotic stimuli, including doxirubicin and camptothecin, where parental and C20 cells were equally sensitive to cell death (data not shown).

Light microscopic observation of MCF-7 cells treated with γTriDAP revealed morphologic changes characteristic of apoptosis and not necrosis (FIG. 2C, panels a-b). However, to confirm that the cell death induced by γTriDAP was indeed apoptosis, DAPI and TUNEL staining was performed. As shown in FIG. 2C, MCF-7 cell nuclei contained condensed chromatin after treatment with γTriDAP, as shown by DAPI staining (panels c-d), with nuclear fragmentation as observed by TUNEL staining (panels e-f). No nuclear staining was detectable in untreated cells.

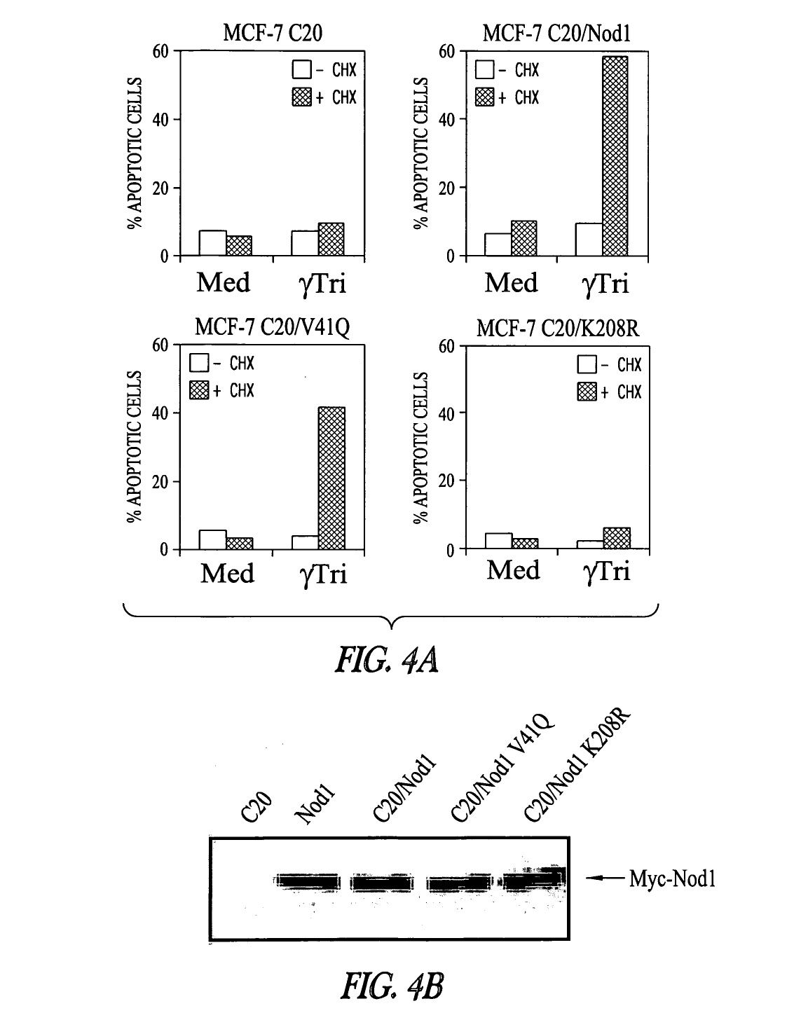

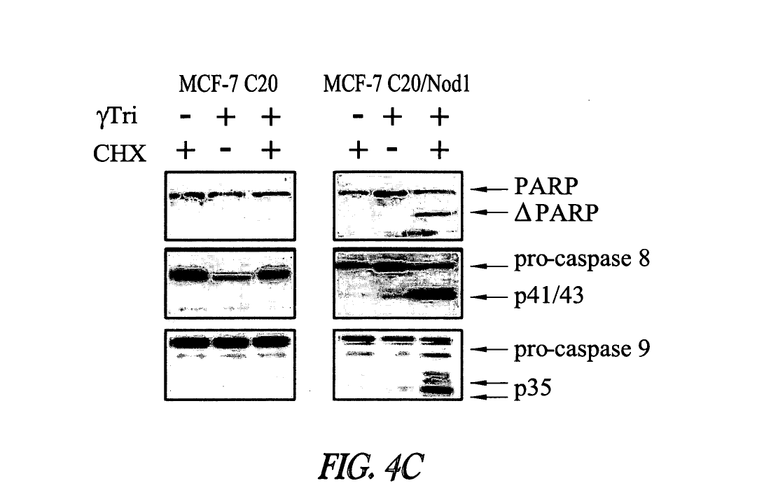

Further confirmation that γTriDAP induces apoptosis was obtained using two broad spectrum caspase inhibitors, z-VAD-FMK and Boc-D-FMK. Both z-VAD-FMK and Boc-D-FMK abrogated γTriDAP-induced cell death (FIG. 2D). Finally, addition of γTriDAP, but not the inactive control tri-peptide αTriDAP, to MCF-7 cells resulted in proteolytic cleavage of poly(ADP-ribose)polymerase (PARP) and capases 6, 7, 8 and 9 (FIG. 3, 9B). MCF-7 cells are known to lack caspase 3, and expression of caspase 3 in parental MCF-7 cells or MCF-7 C20 cells did not change the response patterns to γTriDAP (data not shown).

Thus, multiple lines of evidence indicate that there is a specific apoptotic pathway induced in MCF-7 cells by γTriDAP, the cognate ligand for Nod1, and that this pathway requires the presence of the Nod1 protein.

To further establish that the resistance of MCF-7C20 cells to γTriDAP-induced apoptosis resulted from the absence of Nod1, human Nod1 was stably expressed in MCF-7C20 cells to produce Nod1-expressing MCF-7C20 cells (MCF-7C20/Nod1). Reintroduction of Nod1 into these Nod1-deficient cells restored full sensitivity of these cells to γTriDAP (FIG. 2A), and led to caspase and PARP cleavage (FIG. 3).

To determine if Nod1-dependent apoptosis occurs in other human cell lines, a series of epithelial cell lines were examined where Nod1 was stably expressed to enhance sensitivity to activation of the Nod1 pathway. Cells were treated with TNFα (T) and γTriDAP (γ) in the presence or absence of CHX (C) and monitored for viability by PI staining as a measure of apoptosis (Table 1).

TABLE 1
Cell Viability after TNFα and γTriDAP Treatment

| Cell | Apoptosis | | | | | | | IL-8 Production | | | |
|---|---|---|---|---|---|---|---|---|---|---|---|
| Lines | Ctrl | T | γ | γ/C | T/C | γ/T | γ/T/C | Ctrl | T | γ/C | γ/T |
| SK-BR3 | − | − | − | − | + | − | +++ | − | ++ | ++ | ++ |
| A431 | − | + | − | − | + | − | ++ | − | + | +++ | +++ |
| 293 | − | − | − | − | − | − | − | − | + | +++ | +++ |
| HT29 | − | ++ | − | − | +++ | ++ | +++ | ++ | ++ | ++ | +++ |
| CaCo2 | − | + | − | − | ++ | + | ++ | − | + | − | ++ |

Of the cell lines tested, both SK-BR3 and A431 cells revealed a Nod1-dependent apoptotic pathway. However, in contrast to the MCF-7 line, these cells only undergo apoptosis when γTriDAP was added together with TNFα and CHX. CaCo2 and HT29 cells undergo apoptosis in response to TNFα and CHX but no synergistic effects were observed when the Nod1 pathway was activated by γTriDAP at the same time. Other lines such as 293 cells were resistant to γTriDAP-induced cell death even when TNFα and CHX were added. The lack of Nod1-dependent apoptosis is not a result of an inability of γTriDAP to activate NOD1 because 293 cells that express Nod1 do release IL-8 in response to Nod1 ligand. The HT29 and CaCo2 cells were poorly responsive to γTriDAP in both the apoptosis and IL-8 release assays despite the presence of NOD1 protein, suggesting that one or more positive regulators are absent, or a strong negatively regulatory pathway is present. Despite the complex interactions involved, Nod1-dependent apoptosis is demonstrable in several epithelial cell lines, including the SK-BR3 and A431 cell lines.

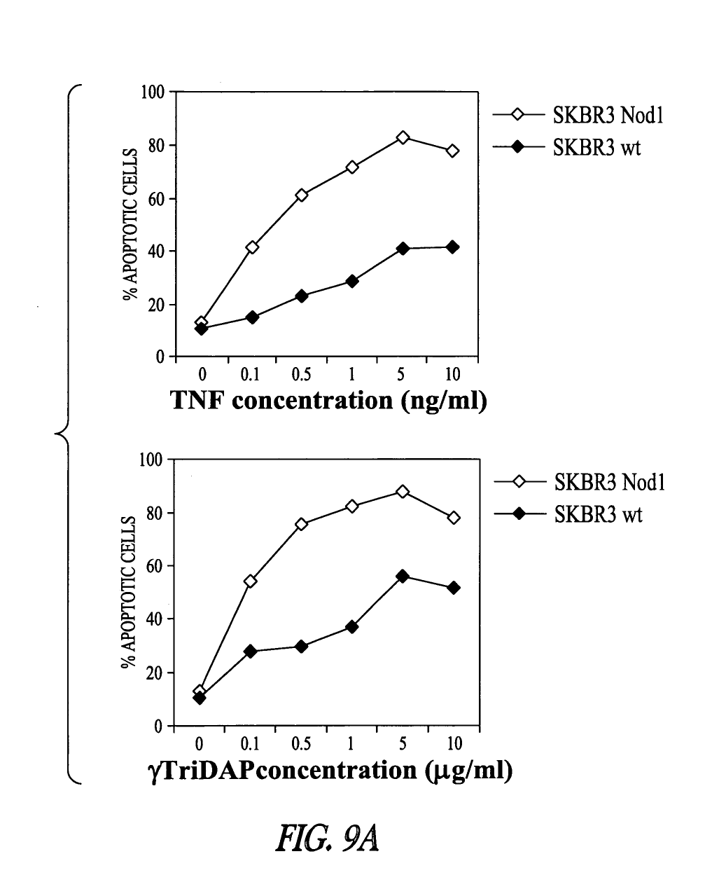

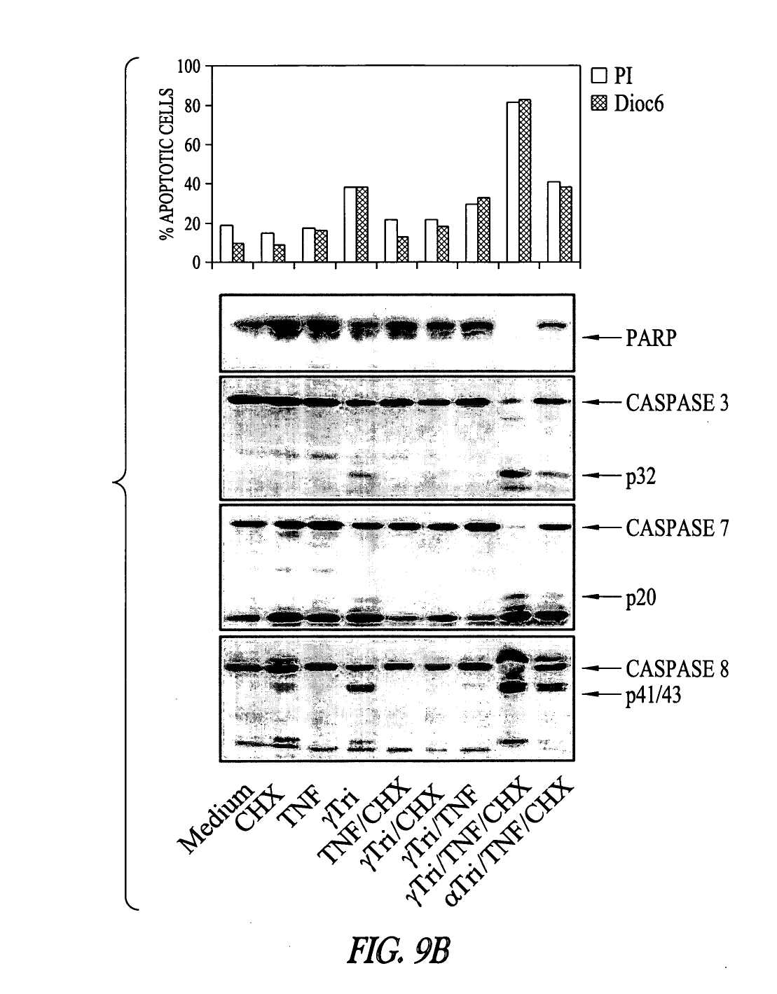

NOD1 is expressed in the SKBR3 human breast cancer cell line and, as indicated above, also modulates apoptosis in those cancer cells. SKBR3 wild type cells exhibit less apoptosis as a function of TNFα or γTriDAP concentration, than SKBR3 cells that over-express Nod1 (FIG. 9A). Moreover, the percentage of apoptotic SKBR3 cells varies depending upon the culture conditions (FIG. 9B). As shown in FIG. 9B, apoptosis of SKBR3 cells is highest when the cells are exposed γTriDAP (γTri) plus TNFα (TNF) and cycloheximide (CHX). Substitution of the inactive γTriDAP analog, αTriDAP (αTri), led to reduced apoptosis. Western analysis of lysates of SKBR3 cells that had been exposed to these agents showed that poly(ADP-ribose)polymerase (PARP) and capases 3, 7, and 8 underwent proteolytic cleavage. FIG. 9C graphically illustrates the percentage of apoptotic wild type, NOD1-expressing and CLARP-expressing SKBR3 cells that were observed after culturing SKBR3 cells in the presence of different agents. As indicated, apoptosis of SKBR3 cells is highest when cycloheximide (CHX or C), TNFα (TNF or T) and γTriDAP (γTri) are present (i.e., the “gTC” combination). However, CLARP expression did not increase apoptosis under a variety of culture conditions.

Previous structure-function studies of human Nod1 indicate that a P-loop residue (K208) is required for transiently over-expressed Nod1 protein to activate NF-kB in 293 cells. Other studies indicate that mutation of K208 blocks conformational changes required for oligomerization mediated by the nucleotide-binding/oligomerization (NBD/NOD) domain. The mutation V41Q in the CARD domain of Nod1 has also been shown to disrupt binding of Caspase 9 to Nod1, resulting in inhibition of Nod1-dependent apoptosis during transient transfection studies in 293 cells.

Further experiments were performed to determine which NOD1 structural domains were needed for apoptosis. As shown in FIG. 4A, the K208R mutation abrogated the effects of γTriDAP plus cycloheximide, while the V41Q mutation had little or no significant effect. These mutant Nod1 polypeptides were efficiently expressed, as shown by the western blot depicted in FIG. 4B. Thus, the different effects of these polypeptides were not due to differences in expression levels. Instead, these data indicate that the nucleotide-binding/oligomerization (NBD/NOD) domain, which mediates the conformational changes required for oligomerization, are needed for Nod1 apoptosis.

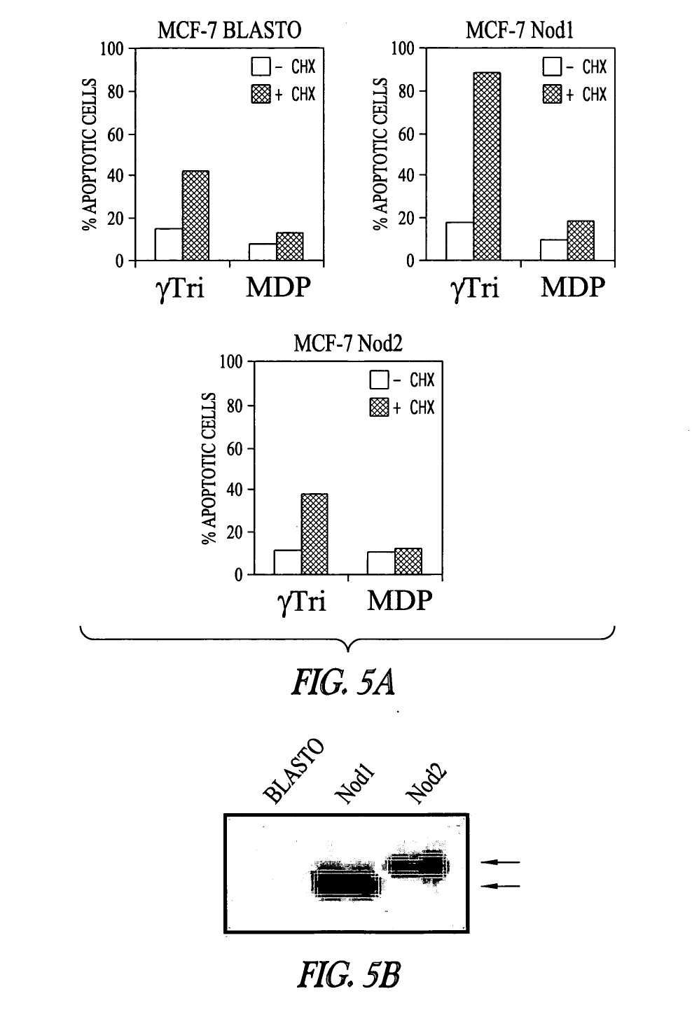

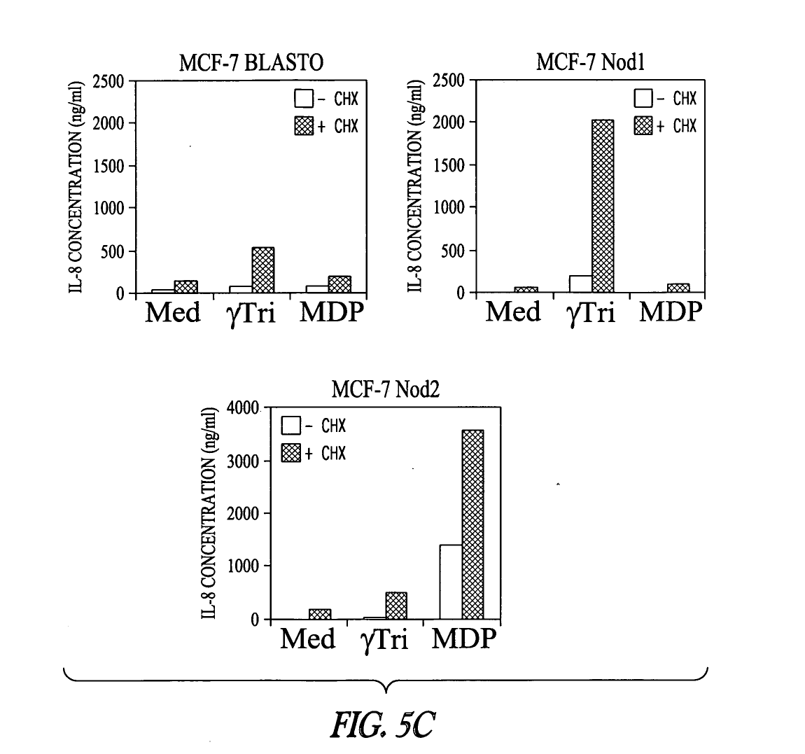

Further experimentation showed that while γTriDAP-induced IL-8 production was inhibited in MCF-7C20 cells (data not shown), such IL-8 production was restored when wild-type Nod1 was expressed in MCF7-C20Nod1 cells (FIG. 5C), or when the V41Q Nod1 mutant was expressed in MCF-7C20 (i.e., MCF-7C20V41Q) cells (data not shown). However, expression of the K208R Nod1 mutant polypeptide in MCF-7C20 (i.e., MCF-7C20K208R) cells did not restore γTriDAP-induced apoptosis.

Nod2 Activation does not Induce Apoptosis in MCF-7 Cells

Experiments were also performed to ascertain whether activation of Nod2 by its specific activator, muramyl dipeptide (MDP), would initiate apoptosis in parental MCF-7 cells or in MCF-7 cells over-expressing Nod2 (MCF-7 Nod2). Thus, γTriDAP or MDP were added to each of these lines and apoptosis (FIG. 5A) and IL-8 production (FIG. 5C) were measured. MCF-7 Nod2 cells treated with MDP plus CHX did not undergo increased apoptosis. In contrast, γTriDAP addition produced cell death, as expected. MDP treatment did result in IL-8 secretion in MCF-7 Nod2 cells (FIG. 5C). Expression of Nod1 and Nod2 in MCF-7 stable transfectants was confirmed by immunoblotting (FIG. 5B). Complementation of MCF-7 C20 cells with Nod2 did not result in apoptosis after addition of either MDP or γTriDAP (data not shown).

Caspase Involvement in Nod1-Dependent Apoptosis

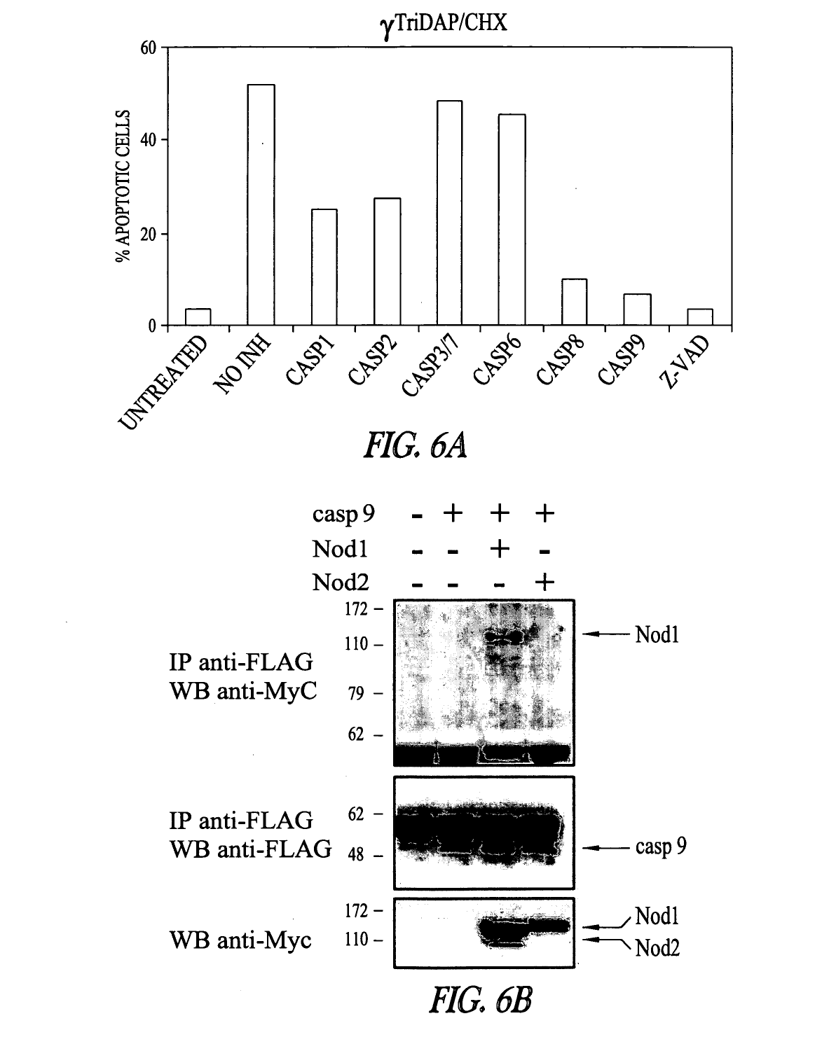

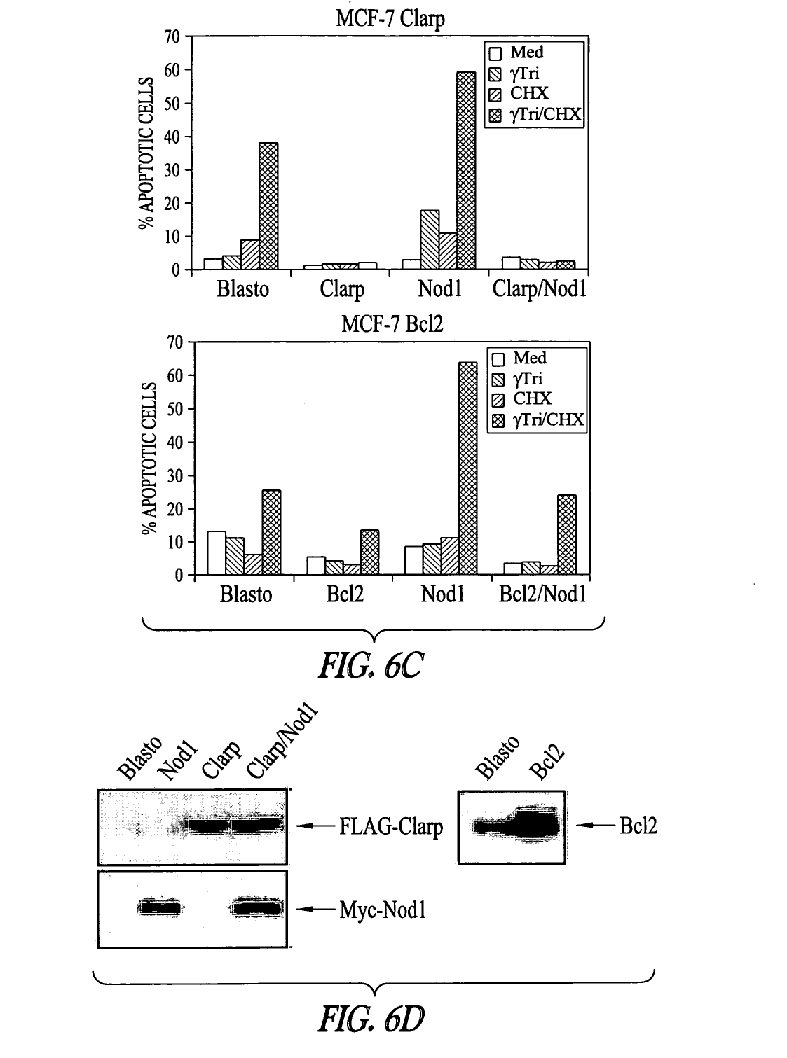

It is now understood that apoptosis occurs following activation of distinct intracellular pathways initiated by specific caspases. To obtain information about the initiating caspases in Nod1-dependent pathways, pharmacologic and biologic inhibitors were used that had varying specificities towards caspases. The broad-spectrum inhibitor z-VAD blocked γTriDAP-induced apoptosis nearly completely (FIG. 6A). In contrast, specific inhibitors of caspases 1, 2, 6, and 7 had only minimal effects on apoptosis (FIG. 6A). However, the caspase 9 inhibitor LEHD and caspase 8 inhibitor IETD had marked inhibitory effects with levels of inhibition similar to those seen with z-VAD (FIG. 6A). These data suggest a possible role for caspase 8 and 9 in the initiation of γTriDAP-induced apoptosis.

Two major apoptotic pathways have been described, the intrinsic (mitochondria, stress induced) and the extrinsic (receptor mediated) pathways. Caspases that participate in apoptosis appear to be organized into hierarchical cascades with caspase 9 and caspase 8 being the upstream initiators in the intrinsic and extrinsic pathways, respectively. To distinguish between these pathways several protein inhibitors were used that were introduced into cells by transfection.

First, CLARP (Flip) transfectants were tested. CLARP is believed to be a specific inhibitor of caspase 8, has two death effector domains (DEDs) and has an inactive caspase domain. CLARP is known to interact with caspase 8 and FADD and thereby specifically inhibit apoptosis induced by various ligand-receptor pairs including Fas, TNFα and TRAIL. To determine the effects of CLARP on γTriDAP-induced cell death, an MCF-7 cell line was established that stably expressed CLARP (MCF-7 CLARP) alone or in the presence of Nod1. When MCF-7CLARP cells were incubated with γTriDAP/CHX, cell death was totally inhibited, suggesting that the Nod1-induced apoptotic pathway overlaps with a pathway initiated by Caspase 8 (FIG. 6C, top panel).

Another anti-apoptotic protein, Bcl-2, has been shown to prevent the release of cytochrome c from the mitochondria thereby blocking activation of the Apaf1/caspase 9 complex. A stable MCF-7 cell line was created that over-expressed Bcl-2 and cell death was assessed in this Bcl-2-expressing MCF-7 cell upon Nod1 activation. As shown in FIG. 6C (bottom panel), overexpression of Bcl-2 only partially prevented γTriDAP-induced cell death.

MCF-7 cells are known to lack Caspase III. Further studies by the inventors determined that expression of Caspase III in the parental MCF-7 or MCF-7C20 cells did not change the response patterns of these cells to the Nod1 ligand γTriDAP (data not shown). These experiments indicate that Caspase 8 plays a predominant role in initiating γTriDAP-induced apoptosis in MCF-7 cells. Additional support, albeit indirect, derived from the finding that the V41Q mutant of Nod1, which fails to interact with Caspase 9, was equivalent to wild-type Nod1 in supporting γTriDAP induced apoptosis (FIG. 4A) and IL8 production (data not shown).

The CARD Domain but not Kinase Activity is Required for Nod1-Induced Apoptosis.

RIP2 is a protein kinase containing a CARD domain. RIP2 has been shown to be important in Nod1 signaling that leads to NF-kB activation (Kobayashi et al. Nature 416: 194-99 (2002); Chin et al. Nature 416: 190-94 (2002)). Binding of RIP2 to Nod1 via CARD-CARD interactions is believed to be essential for NF-kB activation because RIP2 lacking the CARD domain acts as a dominant negative inhibitor of Nod1 signaling.

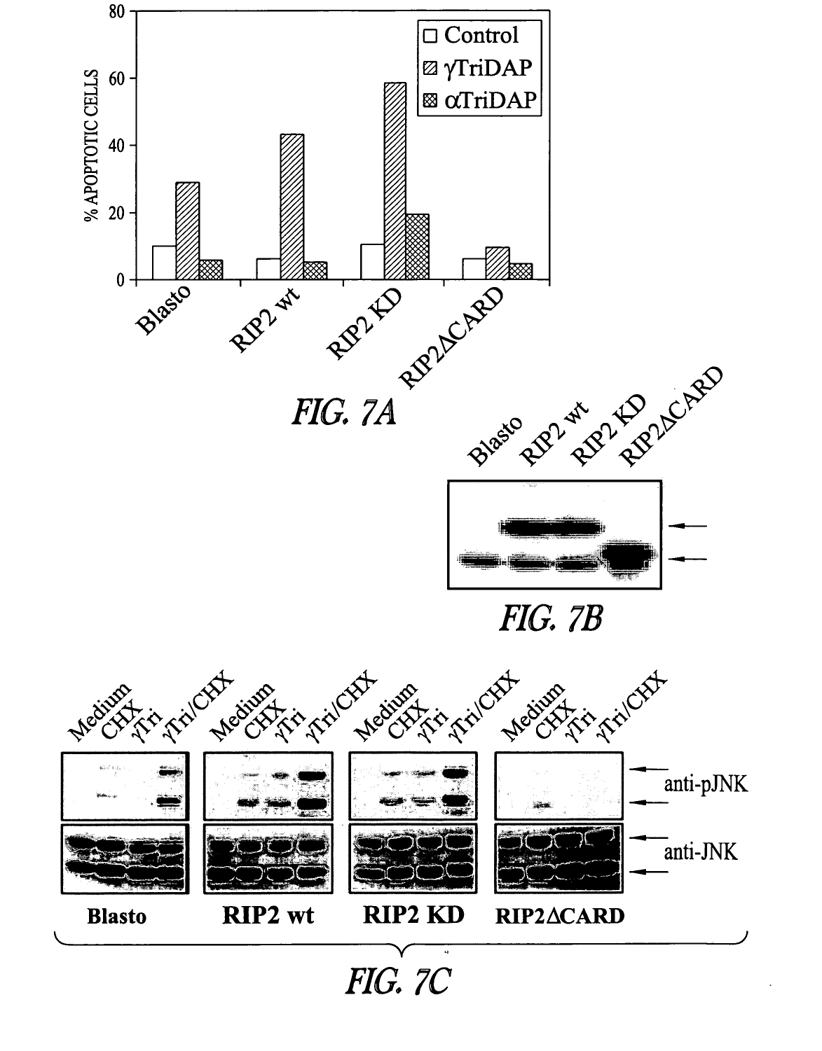

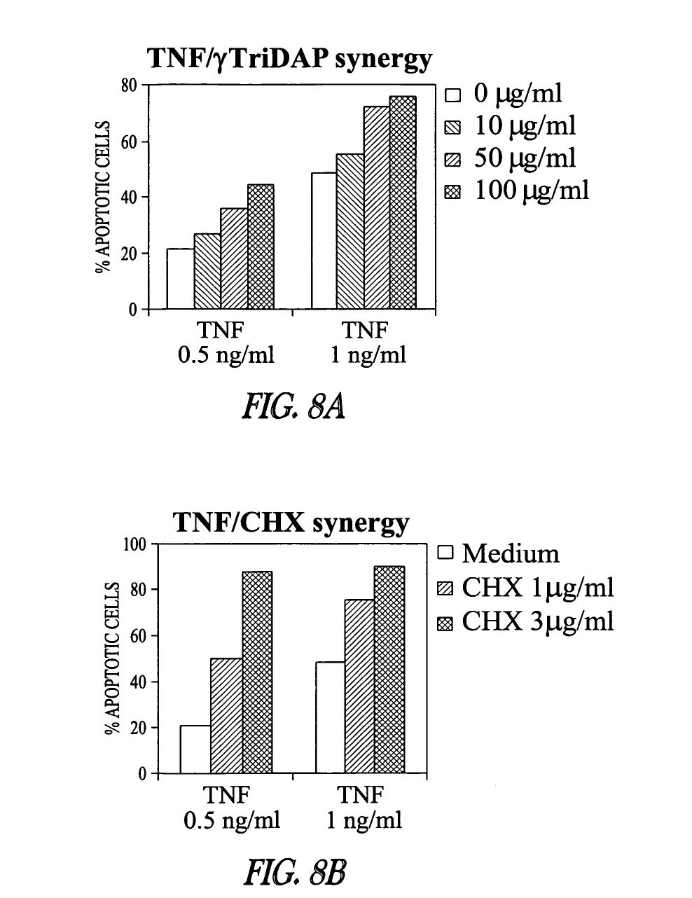

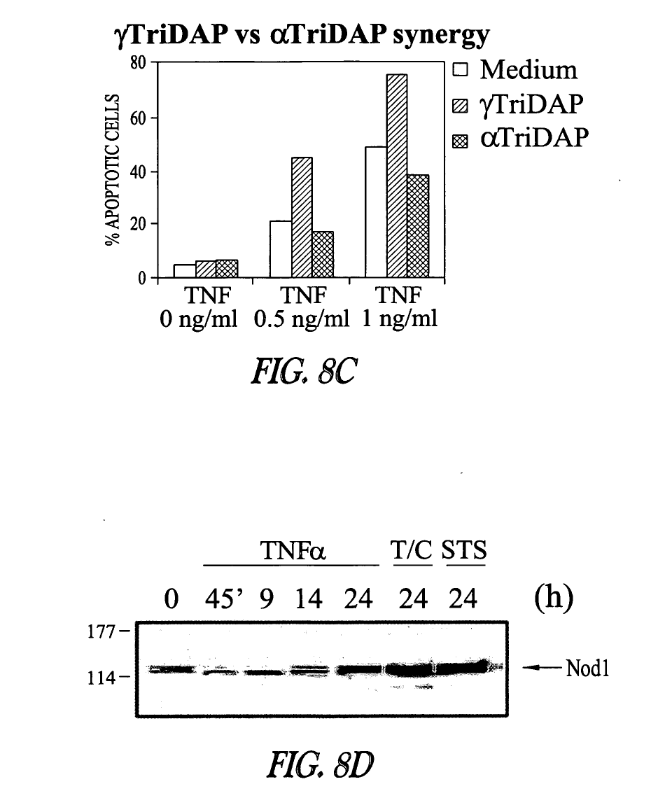

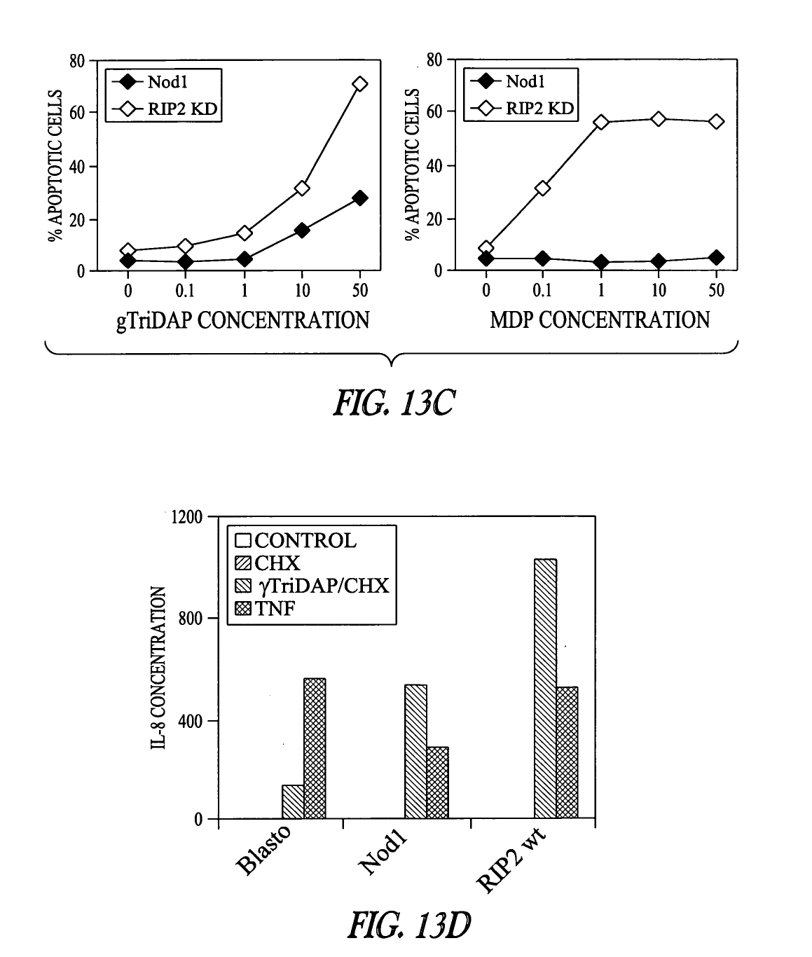

Several RIP2 mutants were stably expressed in MCF-7 cells to evaluate the role of the RIP2 kinase activity in γTriDAP-induced apoptosis. Expression of RIP2 lacking its CARD domain completely abrogated Nod1-induced cell death (FIG. 7A). In contrast, expression of wild type RIP2 or of a catalytically inactive RIP2 (RIP2 KD) increased the extent of apoptosis relative to the apoptosis levels seen in parental MCF-7Blasto cells, which expressed normal levels of Nod1 (FIG. 7A, 13A). Cells expressing wild type RIP2 and RIP2 KD constructs were sensitive to lower concentrations of γTriDAP and died faster than cells expressing only Nod1 (FIG. 13C) or than parental MCF-7Blasto cells (data not shown). Moreover, MDP was effective at inducing high levels of apoptosis in cells expressing RIP2 KD, but not in cells expressing either wild type RIP2 or RIP2ΔCARD (FIG. 13A). MDP also induced higher levels of apoptosis in RIP2 KD cells than in Nod1-espressing cells (FIG. 13C). However, TNFα induced similar levels of apoptosis in cells that expressed wild type RIP2, RIP2 KD and RIP2ΔCARD (FIG. 13B). γTriDAP-induced IL-8 secretion was highest in cells expressing wild type RIP2 (FIG. 13D). Each of the transfected cell lines studied expressed approximately the same amount of wild type or mutant RIP2 (FIG. 7B), indicating that expression levels were not the cause of the differences in cell death. Thus the Nod1-dependent apoptotic pathway requires the RIP2 CARD domain but, surprisingly, does not require RIP2 kinase activity.

The effect of RIP2 expression on γTriDAP-induced JNK phosphorylation was also examined (FIG. 7C). Exposure of MCF-7 cells expressing wild type RIP2 or RIP2 KD to γTriDAP in the presence of cycloheximide for 2 h induced phosphorylation of JNK (FIG. 7C). However, γTriDAP and cycloheximide had no such effect RIP2ΔCARD cells.

Thus Nod1 needs RIP2 (a scaffold protein kinase) for regulating estrogen-sensitive tumor growth. However, while the Nod1-RIP2 regulation of estrogen-sensitive tumor growth requires the RIP2 CARD domain, it does not need RIP2 kinase activity to provide appropriate downstream signals for both Nod1-dependent apoptosis and suppression of tumor growth. Similar results were obtained with p38 (data not shown). The absence of MAPK activation in RIP2ΔCARD cells was not due to altered MAPK kinase signaling because IL-1 strongly induced JNK phosphorylation in all transfectants. Thus, the activity of γTriDAP in MCF-7 cells requires RIP2 but not its kinase activity.

These data indicate that RIP2/RICK, a protein kinase downstream of Nod1, may be an essential component of Nod1 pro-apoptotic pathway because expression of a dominant negative form of RIP2 abolished γTriDAP-induced cell death.

Nod1 controls tumor formation. Nod1-dependent apoptotic pathways might be important in a number of biological processes, including tumor cell growth regulation and a failure of malignant cells to undergo cell death, leading to tumorigenesis. A xenograft model of tumor growth in SCID mice was used to examine the role that Nod1 plays in tumor growth and tumor rejection. Populations of MCF-7Blasto, MCF-7C20 and MCF-7C20Nod1 cells (each totaling about 3×10⁶ cells), were separately injected subcutaneously into the flanks of female mice to induce tumor growth in SCID/SCID or SCID/Nod1 mice. Animals were scored for tumor formation once a week post-injection for up to 8 weeks.

All three cell populations grew tumors equally during the first several weeks after injection so that by 15 days post-injection there was an approximate calculated tumor volume of 10 mm³ in all mice. Thus, MCF-7Blasto, MCF-7C20 and MCF-7C20Nod1 cells could all give rise to tumors by about 15 days after introduction into mice.

However, the tumors formed from MCF-7Blasto and MCF-7C20Nod1 cells almost disappeared over the remainder of the 8 week experiment. The initial tumors produced by MCF-7Blasto and MCF-7C20Nod1 cells quickly diminished in size and regressed (FIG. 10). Only the injection of MCF-7C20 cells produced large round tumors at the site of injection that did not regress and continued to grow. These results indicate that the absence of Nod1 allows for tumor growth, while the presence of Nod1 leads to tumor regression.

Because these studies were performed in SCID mice, the role of an immune system in tumor growth of the xenograft was negligible. Moreover, the absence of tumors in animals receiving MCF-7Blasto and MCF-7C20Nod1 cells was not due to a decreased potential to proliferate, because each of the MCF-7 lines studied here, including cell lines expressing MCF-7Blasto, MCF-7Nod1, MCF-7C20 and MCF-7C20Nod1, had identical growth characteristics in tissue culture conditions and in soft-agar colony forming assays (data not shown).

A summary of results of three separate experiments are shown in Table 2, where SCID/SCID mice were used in Experiments 1 and 2, and SCID/Nod mice were used for experiment 3.

TABLE 2
Incidence of Tumors Induced by MCF-7 Cell Lines

| | Experiment | | | |
|---|---|---|---|---|
| Cell Line | 1 | Experiment 2 | Experiment 3 | Total |
| Wild type MCF-7 | 2/8 | 0/8 | 0/6 | 2/22 |
| MCF-7 C20 | 7/8 | 4/8 | 5/6 | 16/22 |
| MCF-7 C20 Nod1 | 1/8 | 1/8 | 0/6 | 2/22 |

These results indicate that loss of Nod1 function (as when MCF-7 C20 cells were used) increases the probability that tumors will not regress. Replacement of Nod1 function (as when MCF-7 C20 Nod1 cells were used), or induction of Nod1 expression, increases the probability that tumors will regress.

One experiment was also performed where tumors were harvested from tumor-bearing SCID/SCID mice injected 3.5 weeks earlier with MCF-7C20 and MCF-7C20Nod1 cells. The tumor tissue was then minced and placed into tissue culture flasks. After about one week, any remaining solid tumor tissue was removed and 10 μg/ml blasticidin was added. The seeded cells were then maintained under standard conditions for 6 passages in the presence of blasticidin. These cells were then used to inject naïve SCID mice and the time course of changes in tumor volume was measured during a 60 day period.

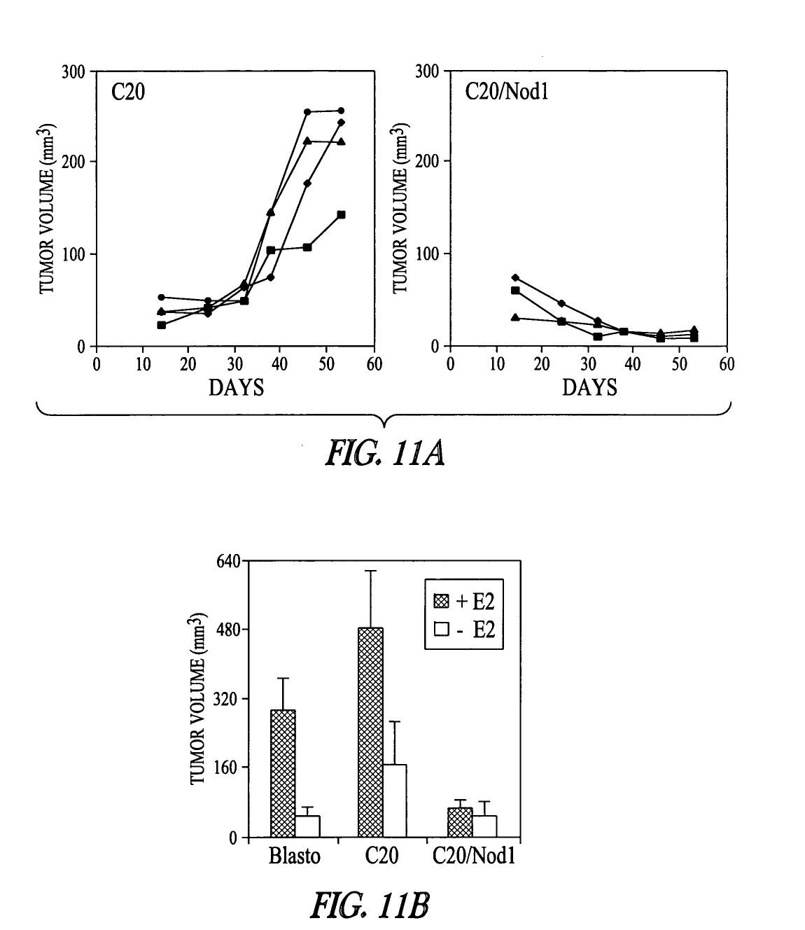

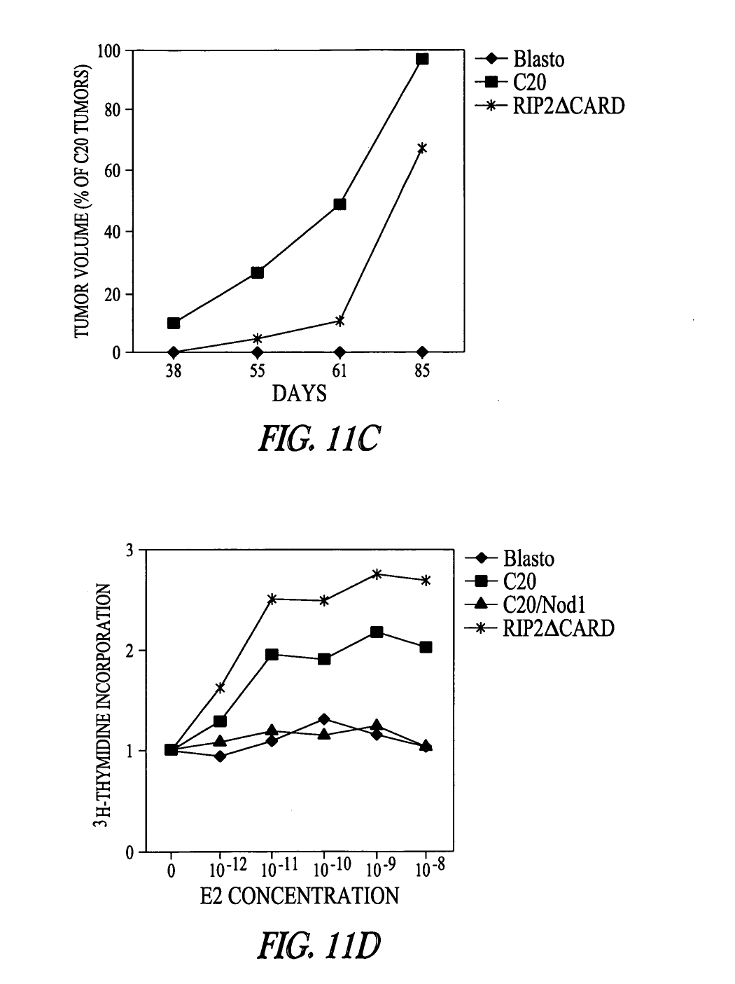

Mice containing either of the tumor-derived MCF-7C20 or MCF-7C20Nod1 cells began to grow tumors. By 15 days after injection both cell types gave rise to an approximate calculated tumor volume of 50 mm³. During the remaining 40 days of this experiment the tumor formed from MCF7-C20Nod1 cells regressed to a minimally detectable size (<10 mm³) while the tumors produced by MCF-7C20 cells grow to a maximum volume of 200-270 mm³ (FIG. 11A-C).

These results further confirm that Nod1 expression in tumor cells can lead to tumor cell apoptosis and tumor regression.

Because the MCF-7 cells undergo apoptosis in response to TNFα, a hamster monoclonal antibody that neutralizes murine TNFα (Bancroft et al. J. Immunol. 143: 127-30 (1989)) was used to further examine the effects of TNFα upon Nod1-induced apoptosis. The presence of this antibody did not inhibit apoptosis because no tumors formed when MCF-7 Blasto or MCF-7 Nod1 cells were inoculated. Moreover, as observed in the previous experiments, tumors did grow when MCF-7 C20 cells were injected into mice.

Again, the absence of tumors after injecting Nod1-expressing tumor cells was not due to a decreased proliferation rate of MCF-7 Blasto and MCF-7 C20/Nod1 cells compared to MCF-7 C20 cells, because each of the MCF-7 lines studied had identical growth characteristics in culture and in soft-agar colony forming assays. Furthermore, simple blockade of apoptosis by other factors was likely not the mechanism that gave rise to tumor growth because CLARP (c-FLIP) is a specific inhibitor of caspase 8, and MCF-7 c-FLIP/CLARP cells were unable to form tumors in nude mice (data not shown).

Role of Estrogen

In preliminary experiments, more robust tumor growth was observed when the C20 clone was transplanted into male mice, suggesting a role for female-related hormones in tumor regression. Further investigation by polymerase chain reaction amplification of a variety of genetic targets, led to the observation that the progesterone receptor was absent in the C20 cell line while it is present in wild-type cells, and more importantly in C20Nod1 cell. Moreover, PCR analysis also revealed that the estrogen receptor alpha was present in each of the three MCF7 cell types.

In most studies, tumor formation by MCF-7 cells requires supplementation of estrogens for tumorigenesis in nude mice, even when cells are inoculated at high concentration. To further examine the role of estrogen in tumorigenesis, all three cell lines were injected into mice along with estrogen pellets (FIG. 11B). As expected tumors grew in mice injected with MCF-7 Blasto cells when estrogen was present. Mice injected with MCF-7 C20 cells produced tumors that grew even larger when estrogen pellets were present. Interestingly, mice that received MCF-7 C20/Nod1 did not grow tumors in the presence of estrogen pellets. These data indicate that Nod1 suppresses estrogen-dependent tumor growth.

To further support the role of Nod1 pathway in tumor growth, expression of a dominant negative allele of RIP2 (RIP2ΔCARD) was investigated to determine whether this allele could also interfere with the ability of MCF-7 cells to grow tumors. FIG. 11C shows that these RIP2ΔCARD cells grew tumors, although the tumors were smaller than the tumors observed in the MCF-7 C20 cells. Taken together, these data indicate that Nod1 has a critical role in tumor growth and that the presence of Nod1 acts as a brake on estrogen-dependent tumor growth.

To obtain additional support for this hypothesis, the sensitivity of the MCF-7 cell lines to estrogen-induced proliferation was observed under conditions where the cells were grown in the absence of estrogen in the culture medium. Under these conditions, the MCF-C20 cells as well as MCF-7 RIP2ΔCARD cells undergo a strong proliferative response to added estrogen while neither the parental nor MCF-7 C20/Nod1 cell lines were stimulated to proliferate (FIG. 11D). However, the estrogen-induced proliferation observed for MCF-7 C20 cells was blocked by addition of tamoxifen in the culture medium (data not shown).

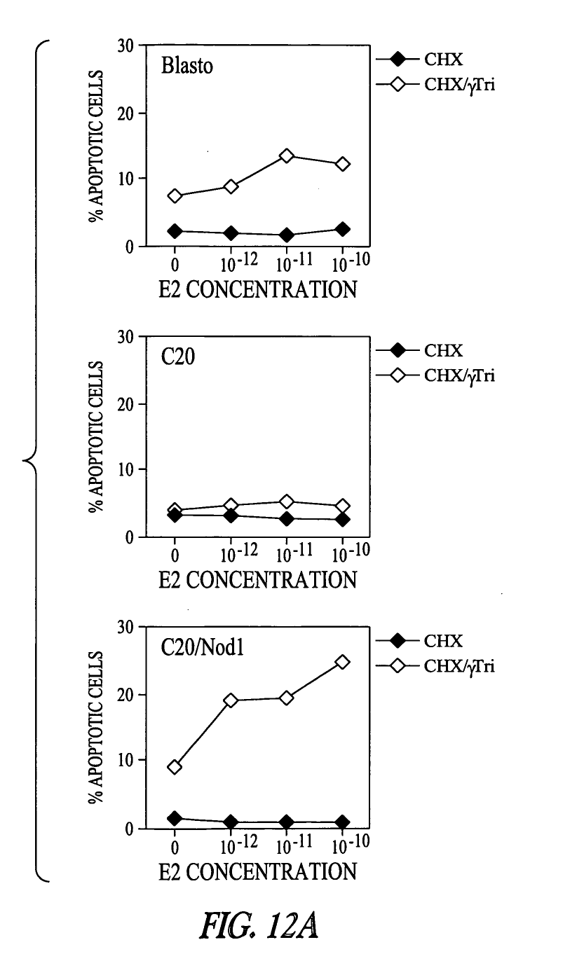

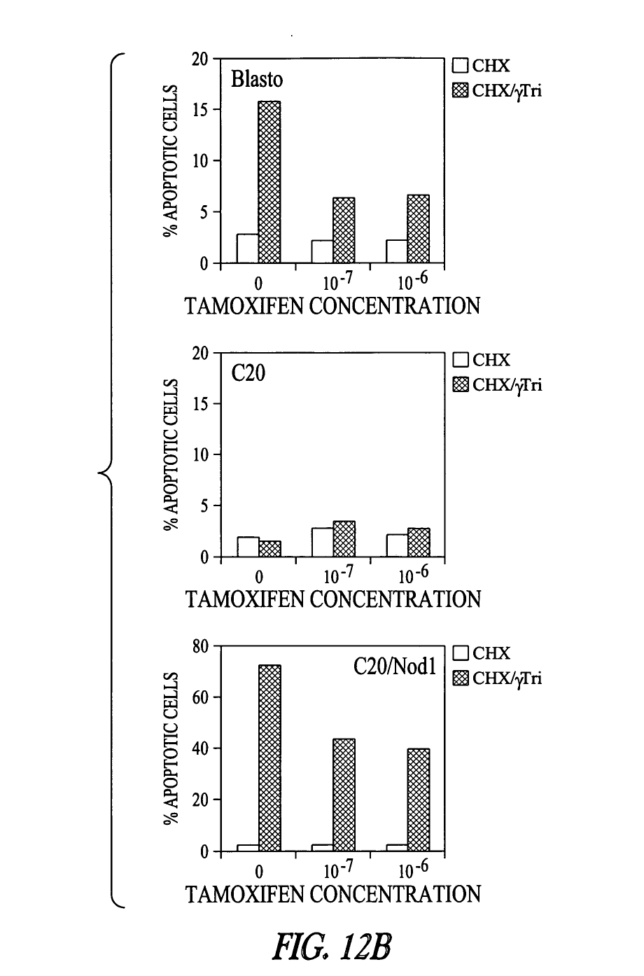

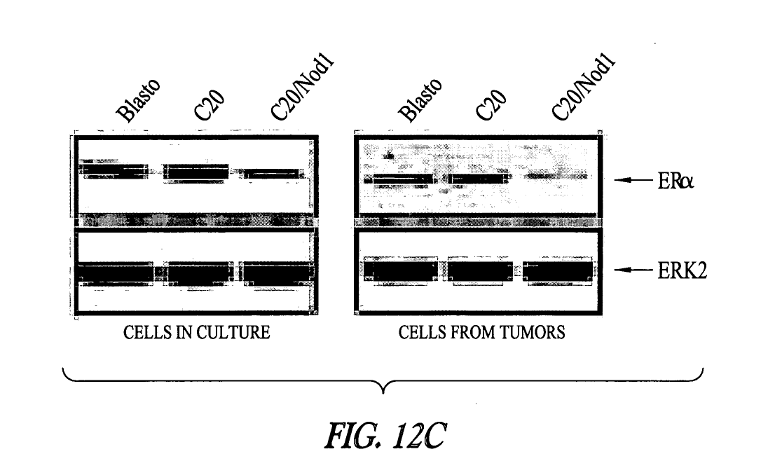

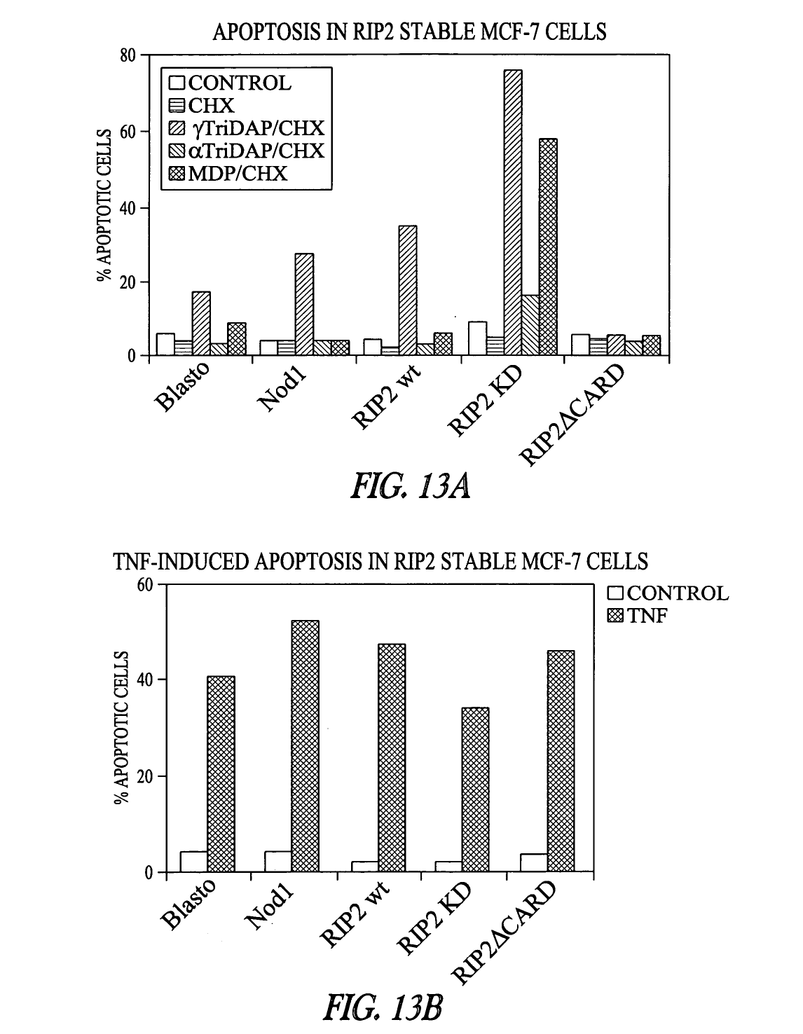

To determine whether the presence of estrogens modulates Nod1-induced apoptotic pathway, cells were cultured in medium containing charcoal-treated serum. Estrogen had little or no effect upon apoptosis in C20 cells, which express substantially no Nod1 and which exhibited substantially no apoptosis (FIG. 12A, middle panel). C20/Nod1 and Blasto cells were more resistant to γTriDAP-induced apoptosis when cultured in the absence of steroids (C20/Nod1 cells exhibited 10% apoptosis without steroids vs 80% with steroids, FIG. 12A, lower panel). Thus, the resistance to apoptosis was reversed by addition of estrogens such that apoptosis increased in a dose-dependent manner with estrogen concentration (FIG. 12A). Conversely, addition of tamoxifen partially blocked γTriDAP-induced cell death (FIG. 12B). Finally, overexpression of Nod1 markedly reduced expression of endogenous estrogen receptor-α (ERα) without affecting that of ERK2, which was used as loading control (FIG. 12C). Similarly, a decrease in ERα expression was observed in re-cultured cells isolated from tumors (FIG. 12C, right panel). These data indicate that Nod1 pathway influences ERα expression levels and therefore the sensitivity of MCF-7 breast cancer cells to develop tumors.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

What is claimed:

1. A composition comprising a carrier, and a therapeutically effective amount of D-Ala-L-Glu-Diaminopimelic acid (γTriDAP), γ-D-glutamy-meso-diaminopimelic acid (iE-DAP), γ-D-Gln-DAP (iQ-DAP), D-Ala-L-Glu-Diaminopimelic acid (γTriDAP), or a combination thereof, wherein the therapeutically effective amount is effective for regression of a tumor.

2. The composition of claim 1, further comprising a nucleic acid that comprises a nucleic acid segment encoding a NOD1 polypeptide.

3. The composition of claim 2, wherein the NOD1 polypeptide comprises SEQ ID NO:1 or SEQ ID NO:3.

4. The composition of claim 2, wherein the segment encoding NOD1 polypeptide comprises SEQ ID NO:2.

5. The composition of claim 2, wherein the nucleic acid further comprises a regulatory element.

6. The composition of claim 2, wherein the regulatory element is a promoter, enhancer, transcriptional termination signal, or a combination thereof.

7. The composition of claim 2, wherein the nucleic acid is an expression cassette or an expression vector.

8. The composition of claim 2, wherein the nucleic acid comprises a gene delivery vehicle.

9. The composition of claim 1, further comprising an effective amount of a NOD1 polypeptide.

10. The composition of claim 1, further comprising an effective amount of tumor necrosis factor α.

11. The composition of claim 1, further comprising an effective amount of cycloheximide.

12. The composition of claim 1, further comprising an effective amount of a hormone or an anti-hormone.

13. The composition of claim 1, further comprising an effective amount of a RIP2 kinase inhibitor.

14. The composition of claim 1, wherein the composition is formulated for local administration to a tumor or a cancerous tissue.

15. A method of promoting tumor regression in a mammal comprising administering to the mammal an agent that increases Nod1 expression or NOD1 activity.

16. The method of claim 15, wherein the tumor is a brain, bladder, cervix, colon, gall bladder, kidney, liver, lung, pancreas, ovary, prostate, skin, stomach, or thyroid tumor.

17. The method of claim 15, wherein the tumor is an estrogen-sensitive tumor.

18. The method of claim 15, wherein the tumor is a breast tumor.

19. The method of claim 15, wherein the agent is D-Ala-L-Glu-Diaminopimelic acid (γTriDAP), γ-D-glutamy-meso-diaminopimelic acid (iE-DAP), γ-D-Gln-DAP (iQ-DAP), D-Ala-L-Glu-Diaminopimelic acid (γTriDAP), or a combination thereof.

20. The method of claim 15, wherein the agent is a nucleic acid that comprises a nucleic acid segment encoding a NOD1 polypeptide.

21. The method of claim 20, wherein the NOD1 polypeptide comprises SEQ ID NO:1 or SEQ ID NO:3.

22. The method of claim 20, wherein the nucleic acid segment encoding NOD1 polypeptide comprises SEQ ID NO:2.

23. The method of claim 20, wherein the nucleic acid further comprises a regulatory element.

24. The method of claim 23, wherein the regulatory element is a promoter, enhancer, transcriptional termination signal, or a combination thereof.

25. The method of claim 20, wherein the nucleic acid is an expression cassette or an expression vector.

26. The method of claim 20, wherein the nucleic acid comprises a gene delivery vehicle.

27. The method of claim 15, wherein the agent is a NOD1 polypeptide.

28. The method of claim 27, wherein the NOD1 polypeptide comprises SEQ ID NO:1 or SEQ ID NO:3.

29. The method of claim 15, wherein the agent is administered locally to the site of the tumor or a cancerous tissue.

30. The method of claim 15, wherein the agent is formulated for sustained release.

31. The method of claim 15, wherein the agent is administered in conjunction with an effective amount of tumor necrosis factor α.

32. The method of claim 15, wherein the agent is administered in conjunction with an effective amount of cycloheximide.

33. The method of claim 15, wherein the agent is administered in conjunction with a RIP2 kinase inhibitor.

34. The method of claim 15, wherein the agent is administered in conjunction with an effective amount of a hormone or an anti-hormone.

35. The method of claim 34, wherein the hormone is estrogen.

36. The method of claim 34, wherein the anti-hormone is an anti-estrogen.

37. The method of claim 15, wherein the agent is administered in conjunction with an effective amount of a chemotherapeutic compound.

38. The method of claim 37, wherein the chemotherapeutic compound is selected from the group consisting of Altretamine, Bleomycin, Busulphan, Calcium Folinate, Capecitabine, Carboplatin, Carmustine, Chlorambucil, Cisplatin, Cladribine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Hydroxyurea, Idarubicin, Ifosfamide, Irinotecan, Liposomal doxorubicin, Lomustine, Melphalan, Mercaptopurine, Methotrexate, Mitomycin, Mitoxantrone, Oxaliplatin, Paclitaxel, Pentostatin, Procarbazine, Raltitrexed, Streptozocin, Tegafur-uracil, Temozolomide, Thiotepa, Tioguanine/Thioguanine, Topotecan, Treosulfan, Vinblastine, Vincristine, Vindesine, Vinorelbine, and a combination thereof.

39. An isolated Nod1^{-/-} cell line lacking NOD1 activity.

40. The isolated Nod1^{-/-} cell line of claim 39, which is also lacking progesterone receptor.

41. The isolated Nod1^{-/-} cell line of claim 39, wherein the cell line comprises MCF-7C20 cells.

42. The isolated Nod1^{-/-} cell line of claim 39, further comprising a recombinant Nod1 allele.

43. A method of identifying a chemotherapeutic agent comprising contacting RIP2 enzyme with a test agent, and determining whether the test agent inhibits RIP2 kinase activity, wherein inhibition of RIP2 activity in the cells by the test agent indicates that the test agent is a chemotherapeutic agent.

44. The method of claim 43, wherein the method is performed in vitro.

45. The method of claim 43, wherein the method is performed in vivo.

46. A method of promoting apoptosis in breast tumor cells comprising contacting the breast tumor cells with an effective amount of D-Ala-L-Glu-Diaminopimelic acid (γTriDAP).

47. A method of identifying a agent that increases apoptosis in a cell comprising contacting a Nod1^{-/-} cell with a test agent, and determining whether the test agent increases apoptosis of the cell.

48. The method of claim 47, wherein the test agent that increase apoptosis is useful as a chemotherapeutic agent.
